(19) United States
(12) Patent Application Publication
(10) Pub. No.: US 2021/0139435 A1
(43) Pub. Date: May 13, 2021

(54) PROSTAGLANDIN ANALOGS AND USES THEREOF

(71) Applicant: Lifex Biolabs, Inc., San Jose, CA (US)

(72) Inventors: Cheolhwan Yoon, Seongnam-si (KR); Hongjun Kang, Seongnam-si (KR); Cheolkyu Han, Seongnam-si (KR); Moonhwan Kim, Seongnam-si (KR); Jeongbeob Seo, Seongnam-si (KR); Jun Yeob Yoo, Singapore (SG); Rajan Sreekanth, Singapore (SG); Ho Sup Yoon, Singapore (SG)

(21) Appl. No.: 17/092,461

(22) Filed: Nov. 9, 2020

Related U.S. Application Data
(60) Provisional application No. 62/931,893, filed on Nov. 7, 2019.

(51) Int. Cl. C07D 241/42; A61P 25/16; C07C 233/81; C07C 235/84; C07C 235/78; C07D 215/38; C07C 69/738

(52) U.S. Cl. CPC C07D 241/42; A61P 25/16; C07C 233/81; C07C 69/738; C07C 235/78; C07D 215/38; C07C 235/84

(57) ABSTRACT

The present invention relates to pharmaceutical composition for the prevention or treatment of a disease, disorder, or condition associated with Nurr1, including, as an active ingredient, a prostaglandin analog or a pharmaceutically acceptable salt thereof, wherein the compound has excellent effects in inducing Nurr1, and thus, can be useful as a pharmaceutical composition for the prevention or treatment of a disease, disorder, or condition associated with Nurr1, in particular, cancer, autoimmune disease such as rheumatoid arthritis, schizophrenia, manic depression and neurodegenerative disease such as Alzheimers disease or Parkinson's disease.

### CROSS-REFERENCE TO RELATED APPLICATION(S)

This application claims the benefit of U.S. patent provisional application No. 62/931,893 filed on Nov. 7, 2019 with the uspto, the disclosures of which are incorporated herein by reference in their entirety.

### TECHNICAL FIELD

The present invention relates to prostaglandin analogs, composition comprising the same for preventing or treating disease, disorder, or condition associated with Nurr1, and uses thereof.

### BACKGROUND ART

Neurons are the basic building block of the nervous system and when they are damaged it results in a range of conditions collectively coined as ‘Neurodegenerative diseases’, leading to ataxias or dementias and finally death. Parkinson's disease (PD) is the second most prevalent neurodegenerative disease affecting approximately 0.3% and 1-2% of the general and aged population, respectively. In 1957, the identification of Dopamine (DA), a vital neurotransmitter in the brain, by Arvid Carlsson and his colleagues was considered as a seminal discovery in the field. In addition to contributing to the finding that DA largely lacks in the brain of PD patients it also led to the clinical breakthrough that levodopa (L-3.4-dihydroxyphenylalanine; L-dopa), the precursor of DA, can be used to significantly improve PD-related mobility impairment, though long-term administration results in the risk of side effects and contraindicated in certain situations.

The progressive and selective loss of A9 DA neurons in the substantia nigra (SN) and the presence of Lewy bodies are two neuropathological hallmarks of PD. This leads to depletion of dopaminergic input in the striatum, typified by resting tremor, rigidity, and bradykinesia. Though the cause and origin of PD remain largely unknown, it is likely affected by both environmental and genetic factors like most other neurodegenerative disorders. Exposure to neurotoxins or other environmental toxins rapidly induces parkinsonian symptoms validating the role of environmental factors on PD. Protein misfolding and aggregation are another important factors directly related to PD pathogenesis, evident from the formation of Lewy bodies which are composed of proteinaceous aggregates including α-synuclein. In general, the ubiquitin-proteasome system which protects cells from misfolded proteins gradually declines with age and this corroborates with the observation that age is a major risk factor for developing PD, or most other neurodegenerative diseases.

Recent studies suggest that neuroinflammation also plays a major role in the pathogenesis of PD. Microglia usually exist as deactivated cells that produce anti-inflammatory and neurotrophic factors, whereas when activated they trigger inflammatory responses as observed within the SN of PD post-mortem tissues. Extracellular α-synuclein when oxidized and nitrated, can also induce microglial activation leading to accelerated degeneration of DA neurons. In addition, increased levels of cytokines were observed in the blood or cerebrospinal fluid of PD patients and animal models. Taken together, chronic neuroinflammation appears to contribute to the pathophysiology of PD and pharmacological intervention of the inflammation pathway could be one of the therapeutic strategies to combat PD. In line with this, it has been shown that chronic use of nonsteroidal anti-inflammatory drugs significantly reduces the risk of PD.

Until now, medication for PD has been symptomatic than cure. Even so, it can only manage the early symptoms while it would be harder to treat those at later-onset stage. Levodopa (L-3,4-dihydroxyphenylalanine) or L-DOPA, has remained the gold standard drug for managing PD for over 40 years, despite the emergence of newer drugs. L-DOPA is a dopamine precursor with the ability to cross the blood-brain barrier and gets converted into dopamine. Long-term administration of the drug may inadvertently lead to further complications in their motor performance coupled with decreased drug efficacy. Peripheral side effects such as nausea and hypotension may also result from L-DOPA administration, but this can be counteracted with the co-administration of Carbidopa, a decarboxylase inhibitor. Other medications include dopamine agonists such as rotigotine and ropinirole or monoamine oxidase B inhibitors such as selegiline and rasagiline. Thus far, the key areas for treatment has been to, (a) increase the amount of DA in the brain, (b) use DA analogs which can mimic DA function in the brain and (c) inhibit enzymes that degrade DA. If any medication proves ineffective, as in advanced stage patients, deep brain stimulation is often considered although the risks from surgery is more serious for elderly patients and is unsuitable for those with co-morbidities.

Over the years, extensive progress has been made in our understanding of how key signaling molecules and transcription factors orchestrate the development of mDA neurons in the mouse brain. Nuclear receptors are ligand-activated transcription factors that regulate genes mainly involved in metabolism and inflammation, and several evidences point toward their role in neurodegenerative diseases. Nurr1, an orphan nuclear receptor belonging to NR4A subfamily comprised of NR4A1, NR4A2, and NR4A3 (also known as Nur77, Nurr1, and Nor1) critically regulates mDA neuron development and survival. Nurr1 knockout resulted in a loss of mDA neurons, indicating that Nurr1 plays an essential role for the development and maintenance of mDA neurons. As a transcriptional factor, Nurr1 activates the expression of multiple genes involved in mDA neuronal phenotypes and survival such as the tyrosine hydroxylase (TH) gene, which is the first and rate-limiting step of DA biosynthesis, aromatic amino acid decarboxylase (AADC), dopamine transporter (DAT), vesicular monoamine transporter (VMAT), and Glial cell line-derived neurotrophic factor (GDNF) c-Ret kinase genes, which regulate the DA neurotransmitter phenotype and survival of mDA neurons. These studies substantiate Nurr1's role in the development, maintenance and survival of mDA neurons. In fact, earlier studies revealed that the expression of Nurr1 is diminished in both aged and PD post-mortem brain tissues. Also, functional mutations and polymorphisms of Nurr1 have been identified in rare cases of familial late-onset forms of PD although their biological significance remains elusive. Taken together, these data strongly suggest that the function of Nurr1 is critically related to the neurodegeneration of DA neurons and its activation may improve PD pathogenesis.

Nurr1, has been classified as “orphan” due to the obscurity of endogenous ligands and have become the most-sought-out target in neuroscience research over the past two decades. Identification of Nurr1 ligands/agonists could pave way for an alternative therapy towards treating PD. In this direction, we aimed toward identifying agonists which can bind to Nurr1 and enhance its transcriptional activation function. Our continued screening efforts resulted in the identification of prostaglandins PGE1 and its metabolite PGA1 as endogenous ligands which can directly bind to Nurr1 and activate it (Rajan, S. et al. Nat Chem Biol 16, 876-886 (2020); U.S. patent application Ser. No. 16/633,741). This was supported by the co-crystal structures of Nurr1-LBD bound PGA1/A2, providing the precise molecular model of their binding interactions (Patent: WO2018056905A1; U.S. patent application Ser. No. 16/334,550). The highlight of these interactions is the covalent bonding, due to Michael addition reaction, formed between Nurr1-LBD's Cys566 sidechain sulphur and the C11 atom in PGA1/A2, along with the re-orientation of the functionally important helix H12. Analysis of the interaction and conformational changes near the ligand binding site, indicating a scope to grow the lead molecule (PGA1/A2) to identify PG analog molecules with enhanced activity. Such an approach is typical for structure-based drug-discovery projects in which the potency of a lead molecule could be enhanced by linking it to fragments which can conceal the nearby pockets/cavities in the protein structure. In this direction, our efforts led to the identification of PG analogs (compound 1 and compound 2) likely to be potent Nurr1 agonists and may serve as relevant ligands that can activate Nurr1 with safe and enhanced therapeutic effects for PD.

### DETAILED DESCRIPTION OF THE INVENTION

### Technical Problem

Therefore, it is an object of the present invention to provide a compound of prostaglandin (PG) analog.

It is an object of the present invention to provide a pharmaceutical composition for the prevention or treatment of disease, disorder, or condition associated with Nurr1, including the prostaglandin analog.

It is an object of the present invention to provide a method for preventing or treating prostaglandin related diseases by administering the prostaglandin analog.

It is an object of the present invention to provide a use of the prostaglandin analog for the prevention or treatment of disease, disorder, or condition associated with Nurr1.

### Technical Solution

### SUMMARY OF THE INVENTION

Parkinson's disease (PD) is a neurodegenerative disorder caused by the progressive and selective degeneration of midbrain dopaminergic (mDA) neurons affecting more than 10 million people worldwide, especially those over the age of 65. The treatments currently available are only symptomatic and there are no treatments that can halt or slow down the progression of the disease process.

Nuclear receptor related 1 protein (Nurr1) is a nuclear receptor essential for the development, maintenance and protection of mDA neurons. Nuclear receptors are ligand-activated transcription factors. Despite attempts to identify natural and endogenous ligands, Nurr1 currently remains an orphan nuclear receptor, because the identity of Nurr1 ligands is elusive.

The extensive screening efforts for natural and synthetic ligands of the inventors of the present invention have led us to identify eicosanoids as potential natural ligands of Nurr1. In particular, it is shown that prostaglandins (PGs), E1 (PGE1), E2 (PGE2), A1 (PGA1) and A2 (PGA2), directly bind to the ligand-binding domain (LBD) of Nurr1 and activate it. Furthermore, the inventors of the present invention have also determined the crystal structures of Nurr1-LBD in complex with PGA1 and PGA2. The structural data and analysis of PGA1/A2 (lead molecules) interaction with Nurr1, provided us the scope to design and develop PG analogs which can bind and activate Nurr1 better than the lead molecule.

The present invention includes the concept for structure-based design of PG analogs, the chemical synthesis of these PG analogs, characterization and identification of the best two analogs that activate Nurr1-mediated transcriptional activity supported by animal studies. The present invention also relates to the use of small-molecule ligands for the treatment of Parkinson's disease mediated by inappropriate Nurr1 activity.

In accordance with an aspect of the present invention, there is provided a prostaglandin analog represented by the following Chemical Formula I or a pharmaceutically acceptable salt, stereoisomer, solvate, polymorph, ester, tautomer, or prodrug thereof.

[chemical structure]

wherein,

X is non-substituted or substituted —(C1-C8)alkyl, —[(C1-C8)alkoxy](C1-C8)alkyl-, —(C1-C8)alkylcarboxylic acid, —(C1-C8)alkylcarboxylester, —(C1-C8)akenyl, [(C1-C8)alkoxy](C1-C8)alkenyl, —(C1-C8)alkenyl acid, —(C1-C8)alkenyl ester, —(C1-C8)alkylamide, or —(C1-C8)alkenylamide;

Y is (C1-C8) alkyl or (C1-C8) alkenyl, which is optionally substituted with one or more substituent(s) selected from the group consisting of hydroxy, oxo, halo, (C1-C6)alkyl, (C1-C6)alkoxy, (C6-C10) aryl, (C6-C10) aryloxy being optionally substituted with (C1-C3) alkyl or halo(C1-C3) alkyl, or (C3-C10) cycloalkyl;

A₁ and A₂ are each independently, CH, CH₂, NH or N;

Z′ is ═O, ═CH₂, [symbol] , or [symbol] .

Z″ is ═O, ═CH₂, [symbol] , or [symbol] , R_{d} is H, (C1-C3) alkyl, (C1-C6)acylcarbonyl or tetrahydropyranyl,

in the Chemical Formula, the notation [symbol] is a single bond or a double bond.

### Advantageous Effects

The novel prostaglandin analogs according to the present invention effectively modulate Nurr1, and therefore they are useful as a therapeutic or prophylactic drug for various disease, disorder, or condition associated with Nurr1 such as cancer, autoimmune disease such as rheumatoid arthritis, schizophrenia, manic depression and neurodegenerative diseases such as Alzheimer's disease, or Parkinson's disease.

### BRIEF DESCRIPTION OF THE DRAWINGS

FIGS. 1A and 1B are the chemical diagrams of the PG analogs Compound 1 (A) and Compound 2 (B) of the present invention, where an identical fragment can be seen attached to the C1 carboxyl end in both these analogs (within broken lines). Compound 1 and Compound 2 are PGE1 and misoprostol analogs, evident from the modifications at the C15 and C16 positions, respectively.

FIG. 1C is a drawing which shows the docking pose revealing the interactions made by the benzylamino phenyl ester fragment in Compound 1, with hydrogen bonds shown in black broken lines and non-polar interactions shown in grey broken lines. An inset showing the surface representation clearly reveals the docking of the fragment into the secondary site.

FIG. 1D is a drawing which shows the interactions stabilizing the methyl and hydroxyl groups at C16 of Compound 2 with nearby protein atoms. The Compound 1 is shown in thin stick mode for reference, wherein the hydroxyl group is seen attached to the carbon C15.

FIG. 2A is a drawing which shows changes in numbers of rotation of the mice of control, 6-OHDA group, 6-OHDA+PGE1 group, 6-OHDA+BSC15 of the present invention group and 6-OHDA+BSC19 of the present invention group.

FIG. 2B is a drawing which shows changes in body weights of the mice of control, 6-OHDA group, 6-OHDA+PGE1 group, 6-OHDA+BSC15 of the present invention group and 6-OHDA+BSC19 of the present invention group.

### DETAILED DESCRIPTION OF THE EMBODIMENTS

Hereinafter, the present invention will be described in detail.

Unless otherwise defined, all technical terms used herein have the same meaning as commonly understood by one of ordinary skill in the art to which this invention belongs. Also, although the invention has been described in conjunction with specific methods and samples, their analogs or equivalents should be within the scope of the present invention. Furthermore, the numerical values set forth herein are considered to include the meaning of “about” unless explicitly stated. All publications and other references mentioned herein are hereby incorporated by reference in their entirety.

The definition of residues used herein is described in detail. Unless otherwise indicated, each residue has the following definition and is used in the sense as commonly understood by one of ordinary skill in the art.

The term “halo” refers to F, Cl, Br, or I, and the term is compatibly used with the term “halogen”.

The term “alkyl” means a linear or branched hydrocarbon aliphatic saturated hydrocarbon group with a single bond, and may include, for example, C₁-C₈ alkyl, specifically C₁-C₆ alkyl, more specifically methyl, ethyl, propyl, isopropyl, n-butyl, isobutyl, sec-butyl, tert-butyl, 1-methylpropyl, pentyl, isopentyl, neopentyl, 1-ethylpropyl, hexyl, isohexyl, 1,1-dimethyl butyl, 2,2-dimethylbutyl, 3,3-dimethylbutyl, 2-ethylbutyl. and the like.

The term “haloalkyl” refers to an alkyl group substituted with one or more halogen atom, and the alkyl group is defined as above. Unless otherwise defined, the haloalkyl refers tofluoromethyl, difluoromethyl, chloromethyl, trifluoromethyl or 2,2,2-trifluoromethyl.

The term “alkoxy” means an oxygen group to which a linear or branched saturated hydrocarbon with a single bond is bonded, and may include, for example, C₁-C₈ alkoxy, specifically C₁-C₆ alkoxy, more specifically methoxy, ethoxy, propoxy, n-butoxy, tert-butoxy, 1-methylpropoxy, and the like.

As used herein, the “alkoxyalkyl” refers to alkyl-O-alkyl group, and the alkyl group is defined as above. The unlimited example is methoxymethyl, ethoxymethyl, methoxyethyl or isopropoxymethyl.

As used herein, the term “hydroxy” or “hydroxyl” alone or in combination with other terms means —OH.

As used herein, “acyl” refers to a group of —C(O)-alkyl, where the alkyl group is as defined above. Examples thereof include, but are not limited to, acetyl, propanoyl, and acrylyl. Acyl groups may or may not be substituted with one or more suitable substituents.

The term “cycloalkyl” means a saturated hydrocarbon ring group with a single bond, and may include, for example, C₃-C₁₀ cycloalkyl depending on the number of carbon atoms, specifically C₃-C₈ cycloalkyl, more specifically cyclopropyl, cyclobutyl, cyclopentyl, cyclohexyl, and the like.

The term “heterocycloalkyl” means a saturated hydrocarbon ring group with a single bond including one or more heteroatoms such as N, O, or S in addition to carbon atoms as ring members. Depending on the number and type of heteroatoms contained in the ring, and the number of carbon atoms, for example, the heterocycloalkyl includes 5- to 12-membered heterocycloalkyl, or 5- to 10-membered heterocycloalkyl containing one or more, specifically, one or more heteroatoms selected from the group consisting of N, O and S, more specifically, aziridine, pyrrolidine, pyrrolidinyl, piperidinyl, piperazinyl, morpholinyl, tetrahydrofuranyl or tetrahydropyranyl, and the like.

The term “aryl” means an aromatic substituent containing at least one ring having a shared pi-electron system, and includes monocyclic or fused ring polycyclic (i.e., rings that share pairs of adjacent carbon atoms) groups. For example, depending on the number of carbon atoms contained in the ring, the aryl is specifically C4-C₁₀ aryl, more specifically C₆-C₁₀ aryl, and still more specifically phenyl, naphthyl, and the like.

The term “heteroaryl” means a monoheterocyclic or polyheterocyclic (e.g., diheterocyclic) aromatic hydrocarbon containing one or more heteroatoms such as N, O, or S in addition to a carbon atom as a ring member. For example, depending on the number and type of heteroatoms contained in the ring, and the number of carbon atoms, the heteroaryl includes C₁-C₁₀ heteroaryl, more specifically, C₁-C₈ heteroaryl, C₂-C₁₀ heteroaryl, or C₂-C₅ heteroaryl, containing one or more, specifically one or more heteroatoms selected from the group consisting of N, O, and S.

Examples of the heteroaryl include furanyl, pyranyl, oxazolyl, isoxazolyl, imidazole, pyridyl, pyrazinyl, pyrimidyl, pyridazinyl, oxadiazolyl, thiadiazolyl, tetrazolyl, triazinyl, triazyl, and the like, but are not limited only thereto.

The term “aryloxy” means a group in which any one carbon forming an aromatic substituent is bonded to oxygen. For example, when oxygen is bonded to a phenyl group, it can be expressed as —O—C₆H, —C₆H₄—O—.

Accordingly, in a first embodiment, the present invention provides a prostaglandin analog represented by the following Chemical Formula I or a pharmaceutically acceptable salt, stereoisomer, solvate, polymorph, ester, tautomer, or prodrug thereof.

[chemical structure]

wherein,

X is non-substituted or substituted —(C1-C8)alkyl, —[(C1-C8)alkoxy](C1-C8)alkyl-, —(C1-C8)alkylcarboxylic acid —(C1-C8)alkylcarboxylester, —(C1-C8)akenyl, [(C1-C8)alkoxy](C1-C8)alkenyl, —(C1-C8)alkenyl acid, —(C1-C8)alkenyl ester, —(C1-C8)alkylamide, or —(C1-C8)alkenylamide;

Y is (C1-C8) alkyl or (C1-C8) alkenyl, which may be optionally substituted with one or more substituents selected from the group consisting of hydroxy, oxo, halo, (C1-C6)alkyl, mono-, di-, or tri-halo(C1-C3) alkyl, (C1-C6)alkoxy, (C6-C10) aryl, (C6-C10) aryloxy and (C3-C10) cycloalkyl, said (C6-C10) aryloxy is optionally substituted with (C1-C3) alkyl, or mono-, di-, or tri-halo(C1-C3) alkyl;

A₁ and A₂ are each independently, CH, CH₂, NH or N;

Z′ is ═O, ═CH₂, [symbol] , or [symbol] ;

Z″ is ═O, ═CH₂, [symbol] , or [symbol] , R_{d} is H, (C1-C3) alkyl, (C1-C6)acylcarbonyl or tetrahydropyranyl,

in the Chemical Formula, the notation [symbol] is a single bond or a double bond.

Preferably, Y may be substituted with at least one hydroxyl or oxo group, and the substituents other than hydroxyl or oxo group.

In second embodiment of the present invention, in the Chemical Formula I,

X may be —(C1-C8) alkyl or —(C1-C8) alkenyl which is optionally substituted with one or more substituents selected from the group consisting of hydroxyl, —(C1-C6) alkoxy, —C(═O)ORₐ, —C(═O)NRₐR_{b}, —NHC(═O)Rc

where Rₐ is H, (C1-C8) alkyl, (C6-C9) aryl, (C6-C9) aryloxy, —NH(C6-C9)aryl, 5- to 12-membered heteroaryl having one or more heteroatom selected from the group consisting of N, O and S, said (C1-C8) alkyl, (C6-C9) aryl, 5- to 12-membered heteroaryl may be optionally substituted with halo, hydroxyl, cyano, nitro, amino, substituted amino, (C1-C6)acyl, —ONO₂, (C1-C8) alkoxy, (C1-C8)alkyl, substituted (C1-C8)alkyl, (C1-C8)haloalkyl, (C3-C7)cycloalkyl, (C1-C8)alkylcarboxy, —NHC(═O)R_{c}, or —C(═O)R_{c}, where R_{c} is (C1-C8) alkyl or (C6-C9) aryl which may be optionally substituted with one or more substituents of halo, CF₃, (C1-C6)acyl, amino, substituted amino, cyano, nitro, (C1-C8)alkyl, substituted (C1-C8)alkyl, (C1-C8)haloalkyl, (C1-C8)alkoxy, (C1-C3)acyloxy, and (C6-C9)aryloxy 5- to 12-membered heterocycloalkyl having one or more heteroatoms selected from the group consisting of N, O and S; and

R_{b} is H or —(C1-C6)alkyl.

Preferably, the substituted amino refers to amino group which may be substituted with (C1-C6)alkyl, and the substituted (C1-C6)alkyl refers to (C1-C6)alkyl group which may be substituted with halo, hydroxyl or (C1-C6)alkyl.

Preferably, Rₐ may be one selected from the group consisting of below substituents:

H, —CH₃, —CH₂CH₃, —CH(CH₃)₂, —CH₂CH₂OH, —CH₂CH(OH)CH₂OH, —CH(CH₂OH)₂, —SO₂CH₃,

[chemical structure]

[chemical structure]

Preferably, R_{b} is H or —(C1-C3)alkyl.

In an another embodiment of the present invention, in the Chemical Formula I,

X may be —(C1-C8)alkyl-OH, —(C1-C8)alkyl-O—(C1-C6)alkyl, —(C1-C8)alkyl-CO₂H, —(C1-C8)alkenyl-CO₂H, —(C1-C8)alkyl-CO₂—(C1-C6)alkyl, —(C1-C8)alkyl-CO₂R², —(C1-C8)alkenyl-CO₂R², —(C1-C8)alkyl-CONR³R⁴, —(C1-C8)alkyl-CONHOR⁴, —(C1-C8)alkenyl-CONR³R⁴, or —(C1-C8)alkyl-CONHOR⁴),

wherein,

R² is optionally substituted (i.e., non-substituted or at least one hydrogen being substituted with (C1-C6) alkyl (e.g., methyl), hydroxyl, dihydroxy, halogen (e.g., mono-, di-, or tri-F or -Cl), (C1-C6)alkoxy (e.g., methoxy), or CF3)) —(C1-C6)alkyl, Ar, CH₂Ar, —Ar—NHCO—Ar, or —Ar—CONH—Ar;

R³ is H or —(C1-C6)alkyl;

R⁴ is H, —(C1-C6)alkyl, Ar, Ar—NHCO—Ar, or Ar—CONH—Ar; and

Ar is a (C6-C10) aryl, 5- to 12 membered mono- or bi-heteroaryl, which is optionally substituted with one or more substituents independently selected from the group consisting of (C1-C6)alkoxy, halogen, (C1-C6)alkyl, and CF₃.

In an another embodiment of the present invention, in the Chemical Formula I,

X may be —CH₂CH₂CH₂CH₂CH₂CH₂OH, —CH₂CH₂CH₂CH₂CH₂CH₂OCH₃, —CH₂CH₂CH₂CH₂CH₂CH₂CO₂H, —CH═CHCH₂CH₂CH₂CO₂H, —CH₂CH₂CH₂CH₂CH₂CO₂CH₃, —CH₂CH₂CH₂CH₂CH₂CO₂CH₂CH₃, —CH₂CH₂CH₂CH₂CH₂CO₂R², —CH═CHCH₂CH₂CH₂CO₂R², —CH₂CH₂CH₂CH₂CH₂CONR³R⁴, —CH₂CH₂CH₂CH₂CH₂CONHOR⁴, —CH═CHCH2CH2CH2CONR³R⁴, or —CH═CHCH₂CH₂CH₂CONHOR⁴.

In an another embodiment of the present invention, in the Chemical Formula I,

Y may be (C1-C6) alkyl or (C1-C6) alkenyl, which may be optionally substituted with one to three substituent(s) selected from the group consisting of hydroxy, oxo, halo, methyl, CF₃, methoxy, phenyl, phenoxy being optionally substituted with CF₃, cylobutyl, and cyclohexyl.

In a specific embodiment, in Formula I,

X may be —CH₂CH₂CH₂CH₂CH₂CO₂R², CH₂CH₂CH₂CH₂CH₂CONR³R⁴, —CH₂CH₂CH₂CH₂CH₂CONHOR⁴, —CH═CHCH₂CH₂CH₂CO₂H, —CH═CHCH2CH2CH2CONR³R⁴, or —CH═CHCH₂CH₂CH₂CONHOR⁴, and

Y may be —(C1-C6)hydroxyalkyl that is non-substituted or substituted with (C1-C6) alkyl (e.g., methyl),

wherein R² and R⁴ may be independently Ar, —Ar—NHCO—Ar, or —Ar—CONH—Ar, and Ar may be a phenyl non-substituted or substituted with halogen (e.g., mono-, di-, or tri-F or -Cl), and

R³ is H or —(C1-C6)alkyl (e.g., H),

with the proviso that if Y is 1-hydroxyhexyl, X is not CH₂CH₂CH₂CH₂CH₂CO₂R² or —CH═CHCH₂CH₂CH₂CO₂R², wherein R² is —Ar—NHCO—Ar and Ar is non-substituted phenyl.

In a specific embodiment, the prostaglandin analog of Chemical Formula I may be represented by one of Chemical Formula II, as below:

[chemical structure]

wherein X, Y and R_{d} are the same as defined in Chemical formula I above.

In an embodiment, the prostaglandin analog of Chemical Formula I may be represented by one of Chemical Formula IIIa, IIIb, IIIc, IIId, IIIe and IIIf, as below:

[chemical structure]

wherein;

R¹ is CH₂OH, CH₂OCH₃, CO₂H, CO₂CH₃, CO₂CH₂CH₃, CO₂R², CONR³R⁴, or CONHOR⁴.

R² is —(C1-C6)alkyl, Ar, —CH₂Ar, —Ar—NHCO—Ar, or —Ar—CONH—Ar, said —(C1-C6)alkyl is optionally substituted with one or more selected from the group consisting of (C1-C6) alkyl, hydroxyl, halogen, (C1-C6)alkoxy, or CF3;

R³ is H or —(C1-C6)alkyl;

R⁴ is H, —(C1-C6)alkyl, Ar, Ar—NHCO—Ar, or Ar—CONH—Ar; and

Ar is a (C6-C10) aryl, 5- to 12 membered heteroaryl, or hetero-biaryl, which is optionally substituted with one or more substituents independently selected from the group consisting of (C1-C6)alkoxy, halogen, (C1-C6)alkyl, and CF₃.

In a specific embodiment, in Formula IIIa and IIIc, R¹ may not be CO₂R², wherein R² may be —Ar—NHCO—Ar, and Ar is a non-substituted phenyl.

In another embodiment, the prostaglandin analog of Chemical Formula I may be represented by one of Chemical Formula IVa, IVb, IVc, and IVd, as below:

[chemical structure]

wherein,

Y is —(C1-C6)alkyl, —(C1-C6)fluoroalkyl, —(C1-C6)difluoroalkyl, —(C1-C6)trifluoroalkyl, —(C1-C6)hydroxyalkyl, —(C2-C6)dihydroxyalkyl, or [(C1-C6)alkoxy](C1-C6)alkyl, which is optionally substituted with one to three substituents selected from the group consisting of (C1-C6) alkyl, halogen, hydroxyl, (C1-C6)alkoxy, or CF₃.

each R⁵ is independently selected from the group consisting of hydrogen, halogen, CF₃, (C1-C6)acyl, amino, substituted amino, (C1-C6)alkyl, substituted (C1-C6)alkyl, (C1-C6)haloalkyl, (C3-C7)cycloalkyl, (C1-C6)alkylcarboxy, cyano, nitro, and (C1-C6) alkoxy,

each R⁶ is independently selected from the group consisting of hydrogen, halogen, CF₃, (C1-C6)acyl, amino, substituted amino, (C1-C6)alkyl, substituted (C1-C6)alkyl, (C1-C6)haloalkyl, cyano, nitro, (C1-C6)alkoxy, (C1-C3)acyloxy, and (C6-C10)aryloxy; and

n is 0, 1, 2, 3, 4 or 5.

Preferably, the substituted amino refers to amino group which may be substituted with (C1-C6)alkyl, and the substituted (C1-C6)alkyl refers to (C1-C6)alkyl group which may be substituted with halo, hydroxyl or (C1-C6)alkyl.

In a specific embodiment, in Formula IVa and IVb, if Y is 1-hydroxyhexyl, both of R⁵ and R⁶ are not hydrogen.

In the formula described herein, a number following “C” refers to the number of carbons; for example, the term “(C1-C6)” refers to comprising 1, 2, 3, 4, 5, or 6 carbons, the term “(C2-C6)” refers to comprising 2, 3, 4, 5, or 6 carbons, and the term “(C3-C7)” refers to comprising 3, 4, 5, 6, or 7 carbons. In the formula described herein, compounds (alkyl, acyl, alkoxy, acyloxy, aryloxy, and the like) without definition of carbon number may be one comprising 1, 2, 3, 4, 5, or 6 carbons, unless it is differently defined.

In the formula described herein, the term “substituted compound” may refer that at least one hydrogen of the compound is independently substituted with other chemical group, for example, one or more, preferably, one to three selected from the group consisting of (C1-C6)alkoxy, halogen, (C1-C6)alkyl, and CF₃, unless it is differently defined.

Further, in a more specific embodiment, the prostaglandin analog of the Chemical Formula I may be one selected from the group consisting of Compounds 2 to 15, 96 and 97, as shown in Table 1 as follows:

TABLE 1

| Compound # | Structure |
|---|---|
| 2 | [chemical structure] |
| 3 | [chemical structure] |
| 4 | [chemical structure] |
| 5 | [chemical structure] |
| 6 | [chemical structure] |
| 7 | [chemical structure] |
| 8 | [chemical structure] |
| 9 | [chemical structure] |
| 10 | [chemical structure] |
| 11 | [chemical structure] |
| 12 | [chemical structure] |
| 13 | [chemical structure] |
| 14 | [chemical structure] |
| 15 | [chemical structure] |
| 96 | [chemical structure] |
| 97 | [chemical structure] |

In a specific embodiment, the prostaglandin analog of Chemical Formula I is not a compound selected from the group consisting of the following compounds shown in Table 2:

TABLE 2

| Compound # | Structure |
|---|---|
| 1 | [chemical structure] |
| 16 | [chemical structure] |
| 17 | [chemical structure] |
| 18 | [chemical structure] |
| 19 | [chemical structure] |
| 20 | [chemical structure] |
| 21 | [chemical structure] |
| 22 | [chemical structure] |
| 23 | [chemical structure] |
| 24 | [chemical structure] |
| 25 | [chemical structure] |
| 26 | [chemical structure] |
| 27 | [chemical structure] |
| 28 | [chemical structure] |
| 29 | [chemical structure] |
| 30 | [chemical structure] |
| 31 | [chemical structure] |
| 32 | [chemical structure] |
| 33 | [chemical structure] |
| 34 | [chemical structure] |
| 35 | [chemical structure] |
| 36 | [chemical structure] |
| 37 | [chemical structure] |
| 38 | [chemical structure] |
| 39 | [chemical structure] |
| 40 | [chemical structure] |
| 41 | [chemical structure] |
| 42 | [chemical structure] |
| 43 | [chemical structure] |
| 44 | [chemical structure] |
| 45 | [chemical structure] |
| 46 | [chemical structure] |
| 47 | [chemical structure] |
| 48 | [chemical structure] |
| 49 | [chemical structure] |
| 50 | [chemical structure] |
| 51 | [chemical structure] |
| 52 | [chemical structure] |
| 53 | [chemical structure] |
| 54 | [chemical structure] |
| 55 | [chemical structure] |
| 56 | [chemical structure] |
| 57 | [chemical structure] |
| 58 | [chemical structure] |
| 59 | [chemical structure] |
| 60 | [chemical structure] |
| 61 | [chemical structure] |
| 62 | [chemical structure] |
| 63 | [chemical structure] |
| 64 | [chemical structure] |
| 65 | [chemical structure] |
| 66 | [chemical structure] |
| 67 | [chemical structure] |
| 68 | [chemical structure] |
| 69 | [chemical structure] |
| 70 | [chemical structure] |
| 71 | [chemical structure] |
| 72 | [chemical structure] |
| 73 | [chemical structure] |
| 74 | [chemical structure] |
| 75 | [chemical structure] |
| 76 | [chemical structure] |
| 77 | [chemical structure] |
| 78 | [chemical structure] |
| 79 | [chemical structure] |
| 80 | [chemical structure] |
| 81 | [chemical structure] |
| 82 | [chemical structure] |
| 83 | [chemical structure] |
| 84 | [chemical structure] |
| 85 | [chemical structure] |
| 86 | [chemical structure] |
| 87 | [chemical structure] |
| 88 | [chemical structure] |
| 89 | [chemical structure] |
| 90 | [chemical structure] |
| 91 | [chemical structure] |
| 92 | [chemical structure] |
| 93 | [chemical structure] |
| 94 | [chemical structure] |
| 95 | [chemical structure] |
| 98 | [chemical structure] |
| 99 | [chemical structure] |
| 100 | [chemical structure] |
| 101 | [chemical structure] |
| 102 | [chemical structure] |

Meanwhile, the compounds of the present invention may exist in the form of a pharmaceutically acceptable salt. As the salt, an addition salt formed by pharmaceutically acceptable free acids may be useful. The term “pharmaceutically acceptable salt” used herein refers to any organic or inorganic addition salt of the prostaglandin analog represented by Chemical Formula I, in which the adverse effect caused by the salt does not impair the beneficial effect of the compound at a concentration exhibiting relatively non-toxic and non-harmful effective activity to a patient.

The acid addition salt may be prepared by a common method, for example, by dissolving a compound in an excess amount of aqueous acid solution and precipitating the resulting salt using a water-miscible organic solvent, such as methanol, ethanol, acetone or acetonitrile. Alternatively, an equimolar amount of a compound and an acid in water or alcohol (e.g., glycol monomethyl ether) can be heated, and subsequently, the resulting mixture can be dried by evaporating, or precipitated salts can be filtered under suction.

In this case, the free acid may be an inorganic acid or an organic acid. Examples of the inorganic acids include, but are not limited to, hydrochloric acid, phosphoric acid, sulfuric acid, nitric acid and stannic acid. Examples of the organic acids include, but are not limited to, methanesulfonic acid, p-toluenesulfonic acid, acetic acid, trifluoroacetic acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, and hydroiodic acid.

In addition, a pharmaceutically acceptable metal salt may be prepared using a base. An alkali metal or alkaline earth metal salt may be obtained, for example, by dissolving a compound in an excess amount of alkali metal hydroxide or alkaline earth metal hydroxide solution, filtering the undissolved compound salt, and then evaporating the filtrate until dry. At this time, as the metal salts, particularly sodium, potassium or calcium salts are pharmaceutically suitable, but the present invention is not limited thereto. Also, the corresponding silver salts may be obtained by reacting an alkali metal or alkaline earth metal salt with a proper silver salt (e.g., silver nitrate).

Pharmaceutically acceptable salts of the compound of the present invention, unless otherwise indicated herein, include salts of acidic or basic groups, which may be present in the compound of Chemical Formula I. For example, the pharmaceutically acceptable salts include sodium, calcium and potassium salts of hydroxy group, and other pharmaceutically acceptable salts of amino group, including hydrobromide, sulfate, hydrogen sulfate, phosphate, hydrogen phosphate, dihydrogen phosphate, acetate, succinate, citrate, tartrate, lactate, mandelate, methanesulfonate (mesylate) and p-toluenesulfonate (tosylate). The salts may be prepared using a salt preparation method known in the art.

Salts of the compounds of Chemical Formula I of the present invention are pharmaceutically acceptable salts, and can be used without particular limitation as long as they are salts of the prostaglandin analogs of Chemical Formula I which can exhibit pharmacological activities equivalent to those of the prostaglandin analog of Chemical Formula I.

In addition, the prostaglandin analogs represented by Chemical Formula I according to the present invention include, but are not limited thereto, not only pharmaceutically acceptable salts thereof, but also all solvates or hydrates and all possible stereoisomers that can be prepared therefrom. All stereoisomers of the present compounds (e.g., those which may exist due to asymmetric carbons on various substituents), including enantiomeric forms and diastereomeric forms, are contemplated within the scope of this invention. Individual stereoisomers of the compounds of the present invention may, for example, be substantially free of other isomers (e.g., as a pure or substantially pure optical isomer having a specified activity), or may be admixed, for example, as racemates or with all other, or other selected, stereoisomers. The chiral centers of the compounds of the present invention may have the S or R configuration as defined by the IUPAC 1974 Recommendations. The racemic forms can be analyzed by physical methods, such as fractional crystallization, separation or crystallization of diastereomeric derivatives or separation by chiral column chromatography. The individual optical isomers can be obtained from the racemates by any suitable method, including without limitation, salt formation with an optically active acid followed by crystallization.

The solvate and stereoisomer of the compound represented by Chemical Formula I may be prepared from the compound represented by Chemical Formula I using methods known in the art.

Furthermore, the prostaglandin analog represented by Chemical Formula I according to the present invention may be prepared either in a crystalline form or in a non-crystalline form, When the compound is prepared in a crystalline form, it may be optionally hydrated or solvated. In the present invention, the compound of Chemical Formula I may not only include a stoichiometric hydrate, but also include a compound containing various amounts of water. The solvate of the compound of Chemical Formula I according to the present invention includes both stoichiometric solvates and non-stoichiometric solvates.

In addition, the above compounds may be used as prodrugs, but is not limited thereto.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis.

In some embodiment, the compounds of the Chemical Formula I of the present invention, for example, Compound 11, but not limited thereto, may have pharmacological effect by themselves, and also act as prodrugs.

Another embodiment provides a pharmaceutical composition for modulating Nurr1, the composition comprising a compound of Chemical Formula I, or a pharmaceutically acceptable salt, stereoisomer, solvate, polymorph, ester, tautomer, or prodrug thereof, as an active ingredient, and a pharmaceutically acceptable carrier. For example, the modulation of Nurr1 may be activation of Nurr1.

Another embodiment provides a method of modulating Nurr1, the method comprising administering an effective amount of a compound of Chemical Formula I, or a pharmaceutically acceptable salt, stereoisomer, solvate, polymorph, ester, tautomer, or prodrug thereof, to a subject in need of modulating Nurr1. For example, the modulation of Nurr1 may be activation of Nurr1. In a specific embodiment, the method may further comprise a step of identifying a subject who is in need of modulating (e.g., activating) Nurr1, before the step of administration.

Another embodiment provides a pharmaceutical composition for preventing or treating a disease, disorder, or condition associated with Nurr1, the composition comprising a prostaglandin analog of Chemical Formula I, or a pharmaceutically acceptable salt, stereoisomer, solvate, polymorph, ester, tautomer, or prodrug thereof, as an active ingredient, and a pharmaceutically acceptable carrier.

Another embodiment provides a method of for preventing or treating a disease, disorder, or condition associated with Nurr1, the method comprising administering an effective amount of a compound of Chemical Formula I, or a pharmaceutically acceptable salt, stereoisomer, solvate, polymorph, ester, tautomer, or prodrug thereof, to a subject in need of preventing or treating a disease, disorder, or condition associated with Nurr1. In a specific embodiment, the method may further comprise a step of identifying a subject who is in need of preventing or treating a disease, disorder, or condition associated with Nurr1, before the step of administering.

The term “subject” is used interchangeably with “individual” and “patient” herein and refers to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage.

In the pharmaceutical composition and the method for preventing or treating a disease, disorder, or condition associated with Nurr1, the disease, disorder, or condition associated with Nurr1 may be disease, disorder, or condition associated with modulated (e.g., inactivated, suppressed, inhibited, ablated, decreased, etc.) Nurr1 (protein and/or gene).

The disease, disorder, or condition may be any one associated with Nurr1 signaling, preferably selected from the group consisting of cancer, autoimmune disease such as rheumatoid arthritis, schizophrenia, manic depression and neurodegenerative diseases such as Alzheimer's disease, or Parkinson's disease, more preferably Parkinson's disease.

Other neurodegenerative diseases that may be treated with a compound or method described herein include Alexander's disease, Alper's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Spinocerebellar ataxia (multiple types with varying characteristics), Spinal muscular atrophy, Steele-Richardson-Olszewski disease, or Tabes dorsalis.

In the pharmaceutical composition and the method provided herein, the compound of Chemical Formula I may be the ones as described above.

In a specific embodiment, the pharmaceutical composition and the method provided herein comprises the compound of Chemical Formula II as described above.

In a specific embodiment, the pharmaceutical composition and the method provided herein comprises the compound of Chemical Formula IIIa, IIIb, IIIc, IIId, IIIe and IIIf as described above.

In a specific embodiment, the pharmaceutical composition and the method provided herein comprises the compound of Chemical Formula IVa, IVb, IVc, and IVd as described above.

In a specific embodiment, the pharmaceutical composition and the method provided herein comprises the compound selected from the group consisting of Compounds 1 to 102 shown above Tables 1 and 2.

The subject may be a mammal including human or a mammalian cell; for example, a mammal (e.g., human) suffering from the disease, disorder, or condition associated with Nurr1 as described above or a mammalian cell isolated therefrom.

The compound as an active ingredient or the pharmaceutical composition may be administered orally or parenterally. For example, the parenteral administration may be performed by any one of intravenous injection, subcutaneous injection, intramuscular injection, intraperitoneal injection, endothelial administration, topical administration, intranasal administration, intrapulmonary administration, intrarectal administration, and the like.

The effective amount may refer to pharmaceutically and/or therapeutically effective amount, and may be prescribed depending on factors such as a type of preparation (formulation), administration route, the patient's age, body weight, gender, and/or pathologic conditions, and the like.

A pharmaceutically acceptable salt of the prostaglandin analog of the present invention may include addition salts formed by inorganic acids such as hydrochloride, sulfate, phosphate, hydrobromide, hydroiodide, nitrate, pyrosulfate, or metaphosphate, addition salts formed by organic acids such as citrate, oxalate, benzoate, acetate, trifluoroacetate, propionate, succinate, fumarate, lactate, maleate, tartrate, glutarate, or sulfonate, or metal salts such as lithium salt, sodium salt, potassium salt, magnesium salt and calcium salt, but is not limited thereto.

The pharmaceutical composition according to the present invention can be formulated into a suitable form together with a commonly used pharmaceutically acceptable carrier. The “pharmaceutically acceptable” refers to being physiologically acceptable, and not usually causing an allergic reaction or a similar reaction such as gastrointestinal disorders and dizziness when administered to humans. Further, the pharmaceutical composition of the present invention may be used after being formulated into an oral preparation, such as powders, granules, tablets, capsules, suspensions, emulsions, syrups, and aerosols, etc., and a parental preparation, such as epidermal formulations, suppositories, or sterile injection solutions, in accordance with a conventional method.

Examples of carriers, excipients and diluents that can be included in the composition, may include lactose, dextrose, sucrose, sorbitol, mannitol, xylitol, erythritol, maltitol, starch, arabic gum, alginate, gelatin, calcium phosphate, calcium silicate, cellulose, methylcellulose, microcrystalline cellulose, polyvinyl pyrrolidone, water, methyl hydroxybenzoate, propyl hydroxybenzoate, talc, magnesium stearate, and mineral oil, but are not limited thereto. When formulated into a preparation, a diluting agent or an excipient, such as commonly-used fillers, stabilizing agents, binding agents, disintegrating agents, and surfactants can be used. Solid preparations for oral administration include tablets, pills, powders, granules, capsules, and the like, and these solid preparations may be prepared by mixing the compound of the present invention with at least one excipient, for example, starch, microcrystalline cellulose, sucrose, lactose, low-substituted hydroxypropyl cellulose, hypromellose or the like. In addition to the simple excipient, a lubricant such as magnesium stearate and talc are also used. Liquid preparations for oral administration include a suspension, a liquid for internal use, an emulsion, a syrup, etc. In addition to a commonly used simple diluent such as water and liquid paraffin, various excipients such as a humectant, a sweetener, an aromatic, a preservative, etc. may also be contained. Formulations for parenteral administration include a sterilized aqueous solution, a non-aqueous solution, a suspension, an emulsion, a lyophilized formulation and a suppository. The non-aqueous solution or suspension may contain propylene glycol, polyethylene glycol, a vegetable oil such as olive oil, an injectable ester such as ethyl oleate, etc. As a base of the suppository, witepsol, macrogol, tween 61, cocoa butter, laurin butter, glycerogelatin, etc. may be used. In order to formulate the formulation for parenteral administration, the compound of Chemical Formula I or a pharmaceutically acceptable salt thereof may be mixed in water together with sterilized and/or contain adjuvants such as preservatives, stabilizers, auxiliary agents such as wettable powder or emulsifying accelerators, salt for controlling osmotic pressure and/or buffers and the like, and other therapeutically useful substances, to prepare a solution or suspension, which is then manufactured in the form of an ampoule or vial unit administration.

The pharmaceutical composition including the compound of Chemical Formula I disclosed herein as an active ingredient may be administered to mammals such as mice, livestock, and humans by various routes for the prevention or treatment of a disease, disorder, or condition associated with Nurr1.

Pharmaceutical formulations described herein are administrable to a subject in a variety of by multiple administration routes, including but not limited to, oral, parenteral (e.g., intravenous, subcutaneous, intramuscular, rectal, enfometrial or cerebrovascular injection), intranasal, buccal, topical or transdermal administration routes.

In some embodiments, the compounds of Chemical Formula I are administered orally.

In some embodiments, the compounds of Chemical Formula I are administered topically.

In another aspect, the compounds of Chemical Formula I are administered by intranasal administration. Such formulations include nasal sprays, nasal mists, and the like.

Intranasal formulations are known in the art. Formulations, which include a compound of Chemical Formula I which are prepared according to these and other techniques well-known in the art are prepared as solutions in saline, employing benzyl alcohol or other suitable preservatives, fluorocarbons, and/or other solubilizing or dispersing agents known in the art. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. Preferably, the nasal dosage form should be isotonic with nasal secretions. The prostaglandin analogs of the present invention may show excellent pharmacological effects for preventing or treating a disease, disorder, or condition associated with Nurr1 when administered via intranasal route.

The dosage is varied depending on the age, sex, weight of the subject to be treated, the specific disease or pathological condition to be treated, the severity of the disease or pathological condition, the duration of administration, the route of administration, the drug absorption, distribution and excretion rate, the types of other drugs used, the judgment of prescriber, and the like. Dosage determination based on such factors is within the standards of those skilled in the art.

Hereinafter, preferred examples of the present invention will be described in detail. However, the present invention is not limited to the examples described herein, and can also be embodied in other forms. Rather, the content presented herein will be thorough and complete, and will fully convey the scope of the present invention to those skilled in the art.

### EXAMPLE

### Preparation Examples

NMR spectra were recorded in CDCl₃ solution in 5-mm o.d. tubes (Norell, Inc. 507-HP) at 30° C. and were collected on Varian VNMRS-400 at 400 MHz for ¹H. The chemical shifts (6) are relative to tetramethylsilane (TMS=0.00 ppm) and expressed in ppm. LC/MS was taken on Ion-trap Mass Spectrometer on FINNIGAN Thermo or ISQ EC, Thermo Fisher U3000 RSLC (Column: YMC Hydrosphere (C18, Ø4.6×50 mm, 3 μm, 120 Å, 40° C.) operating in ESI(+) ionization mode; flow rate=1.0 mL/min. Mobile phase=0.01% heptafluorobutyric acid (HFBA) and 1.0% isopropyl alcohol (IPA) in water or CH₃CN.

[chemical structure]

### Example 1: 4-Benzamidophenyl-7-((1R,2R,3R)-3-hydroxy-2-((S,E)-3-hydroxyoct-1-enyl)-5-oxocyclopentyl)heptanoate (Compound 1)

[chemical structure]

[chemical structure]

To a solution of 7-((1R,2R,3R)-3-hydroxy-2-((S,E)-3-hydroxyoct-1-enyl)-5-oxocyclopentyl)heptanoic acid (10.8 mg, 0.0300 mmol) in DCM (2.0 mL) was added DMAP (3.72 mg, 0.0300 mmol) followed by N-(4-hydroxyphenyl)benzamide (9.10 mg, 0.0430 mmol) at −10° C. under Ar atmosphere. The mixture was stirred for 3 min at −10° C. After addition of EDCI (8.18 mg, 0.0430 mmol), the reaction mixture was stirred at room temperature for 14 hours. The mixture was directly purified by column chromatography on SiO₂ (EtOAc only) to afford 4-benzamidophenyl-7-((1R,2R,3R)-3-hydroxy-2-((S,E)-3-hydroxyoct-1-enyl)-5-oxocyclopentyl) heptanoate (12 mg, 71%) as a white solid.

¹H-NMR (400 MHz, CDCl₃): δ 7.87 (2H, d, J=6.8 Hz), 7.82 (1H, brs), 7.66 (2H, d, J=8.8 Hz), 7.59-7.49 (3H, m), 7.09 (2H, d, J=8.8 Hz), 5.71 (1H, dd, J=15.6, 6.0 Hz), 5.58 (1H, dd, J=15.0, 8.6 Hz), 4.14-4.07 (2H, m), 2.75 (1H, dd, J=18.0, 7.2 Hz), 2.54 (2H, t, J=7.4 Hz), 2.37 (1H, q, J=10.0 Hz), 2.24 (2H, dd, J=18.2, 9.8 Hz), 2.05-1.99 (1H, m), 1.73 (2H, qn, J=7.4 Hz), 1.61-1.26 (17H, m), 0.89 (3H, t, J=6.8 Hz).

### Example 2: 4-Benzamidophenyl-7-((1R,2R,3R)-3-hydroxy-2-((E)-4-hydroxy-4-methyloct-1-en-1-yl)-5-oxocyclopentyl)heptanoate (Compound 2)

[chemical structure]

[chemical structure]

To a solution of DMAP (13.2 mg, 0.109 mmol) in DCM (2.0 mL) was added a solution of 7-((1R,2R,3R)-3-hydroxy-2-((E)-4-hydroxy-4-methyloct-1-enyl)-5-oxocyclopentyl)heptanoic acid (40.0 mg, 0.109 mmol) in methyl acetate (4.0 mL) followed by N-(4-hydroxyphenyl)benzamide (32.4 mg, 0.152 mmol) at −10° C. under Ar atmosphere. The mixture was stirred for 3 min at −10° C. After addition of EDCI (29.1 mg, 0.152 mmol), the reaction mixture was stirred at room temperature for 14 hours. The mixture was directly purified by column chromatography on SiO₂ (EtOAc only) to afford 4-benzamidophenyl-7-((1R,2R,3R)-3-hydroxy-2-((E)-4-hydroxy-4-methyloct-1-en-1-yl)-5-oxocyclopentyl)heptanoate (2) (36 mg, 58%) as a colorless oil.

¹H-NMR (400 MHz, CDCl₃): δ 7.95 (1H, brs), 7.86 (2H, d, J=7.2 Hz), 7.64 (2H, d, J=8.8 Hz), 7.57-7.47 (3H, m), 7.07 (2H, d, J=8.4 Hz), 5.75-5.70 (1H, m), 5.44-5.34 (1H, m), 4.04 (1H, q, J=8.4 Hz), 2.72 (1H, dd, J=18.4, 7.2 Hz), 2.53 (2H, t, J=7.2 Hz), 2.38 (1H, q, J=8.8 Hz), 2.25-2.18 (3H, m), 2.04-1.97 (1H, m), 1.76-1.26 (18H, m), 1.17 (3H, s), 0.91 (3H, t, J=7.2 Hz).

### Example 3: N-(4-(7-((1R,2R,3R)-3-hydroxy-2-((S,E)-3-hydroxyoct-1-en-1-yl)-5-oxocyclopentyl)heptanamido)phenyl)benzamide (Compound 3)

[chemical structure]

To a solution of 7-((1R,2R,3R)-3-hydroxy-2-((S,E)-3-hydroxyoct-1-enyl)-5-oxocyclopentyl) heptanoic acid (10.5 mg, 0.0300 mmol) in DCM (2.0 mL) was added DMAP (3.62 mg, 0.0300 mmol) and EDCI (7.95 mg, 0.041 mmol) at −10° C. under Ar atmosphere. The mixture was stirred for 3 min at −10° C. After addition of N-(4-aminophenyl)benzamide (8.80 mg, 0.0410 mmol), the reaction mixture was stirred at room temperature for 14 hours. The mixture was directly purified by column chromatography on SiO₂ (EtOAc only to EtOAc:MeOH=20:1) to afford N-(4-(7-((1R,2R,3R)-3-hydroxy-2-((S,E)-3-hydroxyoct-1-en-1-yl)-5-oxocyclopentyl)heptanamido) phenyl)benzamide (7.6 mg, 46%) as a white solid.

¹H-NMR (400 MHz, CD₃OD): δ 7.92 (2H, d, J=7.6 Hz), 7.64 (2H, d, J=8.8 Hz), 7.57-7.49 (5H, m), 5.61 (2H, dd, J=6.0, 2.0 Hz), 4.05-4.01 (2H, m), 2.67 (1H, dd, J=20.0, 8.0 Hz), 2.36 (3H, t, J=6.0 Hz), 2.14-2.03 (2H, m), 1.70-1.65 (2H, m), 1.59-1.31 (17H, m), 0.89 (3H, t, J=5.6 Hz).

### Example 4: 7-((1R,2R,3R)-3-hydroxy-2-((S,E)-3-hydroxyoct-1-enyl)-5-oxocyclopentyl)-N-phenylheptanamide (Compound 4)

[chemical structure]

To a solution of 7-((1R,2R,3R)-3-hydroxy-2-((S,E)-3-hydroxyoct-1-enyl)-5-oxocyclopentyl)heptanoic acid (10.0 mg, 0.0280 mmol) in DCM (3.0 mL) was added DMAP (3.45 mg, 0.0280 mmol) followed by aniline (3.68 mg, 0.0390 mmol) at −10° C. under Ar atmosphere. The mixture was stirred at −10° C. for 3 minutes. After addition of EDC (7.57 mg, 0.0390 mmol), the reaction mixture was stirred at room temperature overnight. The mixture was directly purified by column chromatography on SiO₂ (EtOAc only to EtOAc:MeOH=50:1) to afford 7-((1R,2R,3R)-3-hydroxy-2-((S,E)-3-hydroxyoct-1-enyl)-5-oxocyclopentyl)-N-phenylheptanamide (10.6 mg, 87%) as a white solid.

¹H-NMR (400 MHz, CDCl₃): δ 7.72 (1H, brs), 7.51 (2H, d, J=7.2 Hz), 7.32 (2H, t, J=8.0 Hz), 7.17 (1H, brs), 7.10 (1H, t, J=8.0 Hz), 7.40 (1H, dd, J=15.2, 6.0 Hz), 5.60 (1H, dd, J=15.2, 8.8 Hz), 4.29-4.28 (1H, m), 4.17-4.05 (2H, m), 2.76 (1H, dd, J=18.4, 6.8 Hz), 2.41-2.32 (3H, m), 2.24 (1H, dd, J=18.4, 9.6 Hz), 2.05-2.00 (1H, m), 1.73-1.69 (2H, m), 1.49-1.26 (16H, m), 0.89-0.84 (3H, m).

### Example 5: N-(4-chlorophenyl)-7-((1R,2R,3R)-3-hydroxy-2-((S,E)-3-hydroxyoct-1-enyl)-5-oxocyclopentyl)heptanamide (Compound 5)

[chemical structure]

To a solution of 7-((1R,2R,3R)-3-hydroxy-2-((S,E)-3-hydroxyoct-1-enyl)-5-oxocyclopentyl)heptanoic acid (10.0 mg, 0.0280 mmol) in DCM (3.0 mL) was added DMAP (3.45 mg, 0.0280 mmol) and 4-chloroaniline (5.04 mg, 0.0390 mmol) at −10° C. under Ar atmosphere. The mixture was stirred at −10° C. for 3 minutes. After addition of EDC (7.57 mg, 0.0390 mmol), the reaction mixture was stirred at room temperature for 14 hours. The mixture was directly purified by column chromatography on SiO₂ (EtOAc only) to afford N-(4-chlorophenyl)-7-((1R,2R,3R)-3-hydroxy-2-((S,E)-3-hydroxyoct-1-enyl)-5-oxocyclopentyl)heptanamide (9.20 mg, 70%) as a white solid.

¹H-NMR (400 MHz, CDCl₃): δ 7.47 (2H, d, J=8.4 Hz), 7.28 (2H, d, J=10.4 Hz), 7.22 (1H, brs), 5.72 (1H, dd, J=15.6, 6.4 Hz), 5.59 (1H, dd, J=15.2, 8.4 Hz), 4.17-4.05 (2H, m), 2.75 (1H, dd, J=18.8, 6.4 Hz), 2.41-2.31 (3H, m), 2.23 (1H, dd, J=18.4, 9.6 Hz), 2.05-1.99 (1H, m), 1.72-1.66 (2H, m), 1.59-1.25 (18H, m), 0.91-0.87 (3H, m).

### Example 6: 3-(7-((1R,2R,3R)-3-hydroxy-2-((S,E)-3-hydroxyoct-1-enyl)-5-oxocyclopentyl) heptanamido)-N-phenylbenzamide (Compound 6)

[chemical structure]

To a solution of 7-((1R,2R,3R)-3-hydroxy-2-((S,E)-3-hydroxyoct-1-enyl)-5-oxocyclopentyl)heptanoic acid (10.0 mg, 0.0280 mmol) in DCM (3.0 mL) was added DMAP (3.45 mg, 0.0280 mmol) and 3-amino-N-phenylbenzamide (8.38 mg, 0.0390 mmol) at −10° C. under Ar atmosphere. The mixture was stirred at −10° C. for 3 minutes. After addition of EDC (7.57 mg, 0.0390 mmol), the reaction mixture was stirred at room temperature for 14 hours. The mixture was directly purified by column chromatography on SiO₂ (EtOAc only to EtOAc:MeOH=50:1) to afford 3-(7-((1R,2R,3R)-3-hydroxy-2-((S,E)-3-hydroxyoct-1-enyl)-5-oxocyclopentyl) heptanamido)-N-phenylbenzamide (7.0 mg, 45%) as a white solid.

¹H-NMR (400 MHz, CDCl₃): δ 8.46 (1H, brs), 8.10 (1H, brs), 7.66 (3H, t, J=8.0 Hz), 7.61-7.59 (2H, m), 7.41 (1H, t, J=8.0 Hz), 7.37 (2H, t, J=7.6 Hz), 7.15 (1H, t, J=7.2 Hz), 5.71 (1H, dd, J=16.0, 6.4 Hz), 5.59 (1H, dd, J=15.2, 7.6 Hz), 4.11-4.04 (1H, m), 3.67 (1H, s), 2.73 (1H, dd, J=19.2, 7.2 Hz), 2.54 (1H, brs), 2.39-2.19 (4H, m), 2.03-2.00 (1H, m), 2.41-2.32 (3H, m), 1.71-1.10 (16H, m), 0.87-0.84 (3H, m). MS: m/z=531.3 (M+H⁺).

### Example 7: 7-((1R,2R,3R)-3-hydroxy-2-((S,E)-3-hydroxyoct-1-enyl)-5-oxocyclopentyl)-N-(quinoxalin-6-yl)heptanamide (Compound 7)

[chemical structure]

To a solution of 7-((1R,2R,3R)-3-hydroxy-2-((S,E)-3-hydroxyoct-1-enyl)-5-oxocyclopentyl)heptanoic acid (40.0 mg, 0.113 mmol) in DCM (3.0 mL) and THF (1.0 mL) was successively added DIPEA (19.71 μl, 0.113 mmol), HOBT (17.28 mg, 0.113 mmol) and quinoxalin-6-amine (22.9 mg, 0.158 mmol) at −10° C. under Ar atmosphere. The mixture was stirred at −10° C. for 3 minutes. After addition of HATU (42.9 mg, 0.113 mmol), the reaction mixture was stirred at room temperature 14 hours. The mixture was directly purified by column chromatography on SiO₂ (EtOAc:Acetone=10:1 to Acetone only) to afford 7-((1R,2R,3R)-3-hydroxy-2-((S,E)-3-hydroxyoct-1-enyl)-5-oxocyclopentyl)-N-(quinoxalin-6-yl)heptanamide (27 mg, 50%) as a yellow oil.

¹H-NMR (400 MHz, CDCl₃): δ 8.80 (1H, d, J=1.6 Hz), 8.76 (1H, d, J=2.0 Hz), 8.30 (1H, brs), 8.07-8.01 (2H, m), 7.91-7.86 (1H, m), 5.73 (1H, dd, J=15.2, 6.8 Hz), 5.60 (1H, dd, J=11.2, 8.8 Hz), 4.18-4.05 (2H, m), 2.74 (1H, dd, J=18.4, 6.8 Hz), 2.45-2.34 (3H, m), 2.25 (1H, dd, J=18.4, 10.0 Hz), 2.05-2.00 (1H, m), 2.05-2.00 (1H, m), 1.79-1.26 (19H, m), 0.87 (3H, t, J=6.8 Hz).

### Example 8: 7-((1R,2R,3R)-3-hydroxy-2-((S,E)-3-hydroxyoct-1-enyl)-5-oxocyclopentyl)-N-(quinolin-7-yl)heptanamide (Compound 8)

[chemical structure]

To a solution of 7-((1R,2R,3R)-3-hydroxy-2-((S,E)-3-hydroxyoct-1-enyl)-5-oxocyclopentyl)heptanoic acid (20.0 mg, 0.0560 mmol) in DCM (3.0 mL) and THF (1.0 mL) was successively added DIPEA (9.85 μL, 0.0560 mmol), HOBT (8.64 mg, 0.0560 mmol) and quinolin-7-amine (24.40 mg, 0.169 mmol) at −10° C. under Ar atmosphere. The mixture was stirred at −10° C. for 3 minutes. After addition of HATU (32.2 mg, 0.0850 mmol), the reaction mixture was stirred at room temperature for 14 hours. The mixture was directly purified by column chromatography on SiO₂ (EtOAc only to EtOAc:Acetone=10:1) to afford 7-((1R,2R,3R)-3-hydroxy-2-((S,E)-3-hydroxyoct-1-enyl)-5-oxocyclopentyl)-N-(quinolin-7-yl)heptanamide (21 mg, 77%) as a yellow solid.

¹H-NMR (400 MHz, CDCl₃): δ 8.85 (1H, d, J=1.6 Hz), 8.13 (1H, d, J=7.6 Hz), 8.07-8.02 (2H, m), 7.86 (1H, brs), 7.79 (1H, d, J=8.4 Hz), 7.34 (1H, dd, J=8.0, 4.0 Hz), 5.74 (1H, dd, J=15.6, 6.8 Hz), 5.62 (1H, dd, J=15.6, 7.6 Hz), 4.18-4.05 (2H, m), 2.75 (1H, dd, J=18.4, 7.2 Hz), 2.44-2.36 (3H, m), 2.22 (1H, dd, J=18.4, 10.0 Hz), 2.05-2.01 (1H, m), 1.77-1.25 (20H, m), 0.86 (3H, t, J=7.2 Hz).

### Example 9: 7-((1R,2R,3R)-3-hydroxy-2-((S,E)-3-hydroxyoct-1-enyl)-5-oxocyclopentyl)-N-phenoxyheptanamide (Compound 9)

[chemical structure]

To a solution of 7-((1R,2R,3R)-3-hydroxy-2-((S,E)-3-hydroxyoct-1-enyl)-5-oxocyclopentyl)heptanoic acid (25.0 mg, 0.0710 mmol) in DCM (3.0 mL) and THF (1.0 mL) was successively added DIPEA (12.3 μL, 0.0710 mmol), HOBT (10.8 mg, 0.0710 mmol) and O-phenylhydroxylamine (23.1 mg, 0.212 mmol) at −10° C. under Ar atmosphere. The mixture was stirred at −10° C. for 3 minutes. After addition of HATU (40.2 mg, 0.106 mmol), the reaction mixture was stirred at room temperature for 14 hours. The mixture was directly purified by column chromatography on SiO₂ (EtOAc:Acetone=10:1 to Acetone only) to afford 7-((1R,2R,3R)-3-hydroxy-2-((S,E)-3-hydroxyoct-1-enyl)-5-oxocyclopentyl)-N-phenoxyheptanamide (27 mg, 88%) as a yellow oil.

¹H-NMR (400 MHz, CDCl₃): δ 8.84 (1H, brs), 7.32 (2H, brs), 7.07-7.05 (3H, m), 5.68 (1H, dd, J=12.8, 6.8 Hz), 5.55 (1H, dd, J=15.6, 8.8 Hz), 4.13-4.01 (2H, m), 2.73 (1H, dd, J=18.4, 7.6 Hz), 2.39-2.18 (4H, m), 2.02-1.96 (1H, m), 1.64-1.26 (20H, m), 0.88 (3H, t, J=7.0 Hz).

### Example 10: 7-((1R,2R,3R)-3-hydroxy-2-((E)-4-hydroxy-4-methyloct-1-enyl)-5-oxocyclopentyl)-N-phenoxyheptanamide (Compound 10)

[chemical structure]

To a solution of 7-((1R,2R,3R)-3-hydroxy-2-((E)-4-hydroxy-4-methyloct-1-enyl)-5-oxocyclopentyl)heptanoic acid (20.0 mg, 0.0540 mmol) in DCM (3.0 mL) and THF (1.0 mL) was successively added DIPEA (11.4 μl, 0.0650 mmol), HOBT (8.31 mg, 0.0540 mmol) and O-phenylhydroxylamine (7.11 mg, 0.0650 mmol) at −10° C. under Ar atmosphere. The mixture was stirred at −10° C. for 3 minutes. After addition of HATU (28.9 mg, 0.0760 mmol), the reaction mixture was stirred at room temperature for 14 hours. The mixture was directly purified by column chromatography on SiO₂ (EtOAc:Acetone=10:1 to Acetone only) to afford 7-((1R,2R,3R)-3-hydroxy-2-((E)-4-hydroxy-4-methyloct-1-enyl)-5-oxocyclopentyl)-N-phenoxyheptanamide (18 mg, 72%) as a yellow oil.

¹H-NMR (400 MHz, CDCl₃): δ 8.97 (1H, brs), 7.32-7.30 (2H, brs), 7.07-7.05 (3H, m), 5.78-5.70 (1H, m), 5.45-5.39 (1H, m), 4.05 (1H, q, J=8.4 Hz), 2.74 (1H, dd, J=16.0, 7.2 Hz), 2.41-2.05 (7H, m), 2.05-1.98 (2H, m), 1.63-1.26 (16H, m), 1.17 (3H, s), 0.91-0.83 (3H, m).

### Example 11: 4-(4-fluorobenzamido)phenyl 7-((1R,2R,3R)-3-hydroxy-2-((E)-3-hydroxyoct-1-enyl)-5-oxocyclopentyl)heptanoate (Compound 11)

[chemical structure]

To a solution of 7-((1R,2R,3R)-3-hydroxy-2-((E)-3-hydroxyoct-1-enyl)-5-oxocyclopentyl)heptanoic acid (40.0 mg, 0.113 mmol) in DCM (4.0 mL) were successively added 4-fluoro-N-(4-hydroxyphenyl)benzamide (36.5 mg, 0.158 mmol) and DMAP (13.8 mg, 0.113 mmol) at −10° C. under Ar atmosphere. The mixture was stirred at −10° C. for 3 minutes. After addition of EDC (30.3 mg, 0.158 mmol), the reaction mixture was stirred at room temperature for 14 hours. The mixture was purified by column chromatography on SiO₂ (EtOAc only) to afford 4-(4-fluorobenzamido)phenyl 7-((1R,2R,3R)-3-hydroxy-2-((E)-3-hydroxyoct-1-enyl)-5-oxocyclo-pentyl)heptanoate (45.0 mg, 70%) as a white solid.

¹H-NMR (400 MHz, CDCl₃): δ 7.89 (2H, dd, J=8.8, 5.2 Hz), 7.76 (1H, brs), 7.63 (2H, d, J=8.8 Hz), 7.48 (2H, t, J=8.4 Hz), 7.10 (2H, d, J=9.2 Hz), 5.71 (1H, dd, J=14.4, 6.4 Hz), 5.58 (1H, dd, J=14.4, 8.4 Hz), 4.15-4.05 (2H, m), 2.75 (1H, dd, J=14.4, 7.2 Hz), 2.54 (2H, t, J=7.2 Hz), 2.41-2.34 (1H, m), 2.24 (1H, dd, J=18.4, 9.6 Hz), 2.05-1.99 (1H, m), 1.73 (2H, q, J=6.4 Hz), 1.58-1.25 (18H, m), 0.88 (3H, t, J=6.4 Hz).

### Example 12: 4-(3,4-difluorobenzamido)phenyl 7-((1R,2R,3R)-3-hydroxy-2-((S,E)-3-hydroxyoct-1-enyl)-5-oxocyclopentyl)heptanoate (Compound 12)

[chemical structure]

To a solution of 7-((1R,2R,3R)-3-hydroxy-2-((E)-3-hydroxyoct-1-enyl)-5-oxocyclopentyl)heptanoic acid (40.0 mg, 0.113 mmol) in DCM (4.0 mL) was successively added 3,4-difluoro-N-(4-hydroxyphenyl)benzamide (39.4 mg, 0.158 mmol) and DMAP (13.8 mg, 0.113 mmol) at −10° C. under Ar atmosphere. The mixture was stirred at −10° C. for 3 minutes. After addition of EDC (30.3 mg, 0.158 mmol), the reaction mixture was stirred at room temperature for 14 hours. The mixture was purified by column chromatography on SiO₂ (EtOAc only) to afford 4-(3,4-difluorobenzamido)phenyl 7-((1R,2R,3R)-3-hydroxy-2-((S,E)-3-hydroxyoct-1-enyl)-5-oxocyclopentyl)heptanoate (42.3 mg, 64%) as a white solid.

¹H-NMR (400 MHz, CDCl₃): δ 7.77-7.72 (2H, m), 7.62 (2H, d, J=9.2 Hz), 7.32-7.28 (1H, m), 7.10 (2H, d, J=9.2 Hz), 5.71 (1H, dd, J=14.7, 6.4 Hz), 5.58 (1H, dd, J=14.4, 8.0 Hz), 4.14-4.05 (2H, m), 2.75 (1H, dd, J=14.4, 7.2 Hz), 2.55 (2H, t, J=7.2 Hz), 2.45-2.34 (2H, m), 2.24 (1H, dd, J=18.4, 9.6 Hz), 2.05-2.01 (1H, m), 1.84 (1H, brs), 1.73 (2H, qn, J=6.8 Hz), 1.58-1.25 (17H, m), 0.89 (3H, t, J=6.8 Hz).

### Example 13: 4-(3,5-difluorobenzamido)phenyl 7-((1R,2R,3R)-3-hydroxy-2-((S,E)-3-hydroxyoct-1-enyl)-5-oxocyclopentyl)heptanoate (Compound 13)

[chemical structure]

To a solution of 7-((1R,2R,3R)-3-hydroxy-2-((E)-3-hydroxyoct-1-enyl)-5-oxocyclopentyl)heptanoic acid (40.0 mg, 0.113 mmol) in DCM (4.0 mL) was successively added 3,5-difluoro-N-(4-hydroxyphenyl)benzamide (39.4 mg, 0.158 mmol) and DMAP (13.8 mg, 0.113 mmol) at −10° C. under Ar atmosphere. The mixture was stirred at −10° C. for 3 minutes. After addition of EDC (30.3 mg, 0.158 mmol), the reaction mixture was stirred at room temperature for 14 hours. The mixture was purified by column chromatography on SiO₂ (EtOAc only) to afford 4-(3,5-difluorobenzamido)phenyl 7-((1R,2R,3R)-3-hydroxy-2-((S,E)-3-hydroxyoct-1-enyl)-5-oxocyclopentyl)heptanoate (45.3 mg, 69%) as a white solid.

¹H-NMR (400 MHz, CDCl₃): δ 7.77 (1H, s), 7.63 (2H, d, J=9.2 Hz), 7.40 (2H, d, J=6.4 Hz), 7.11 (2H, d, J=9.2 Hz), 7.04-6.99 (1H, m), 5.73 (1H, dd, J=15.6, 6.4 Hz), 5.58 (1H, dd, J=14.4, 8.4 Hz), 4.14-4.05 (2H, m), 2.76 (1H, dd, J=14.4, 6.8 Hz), 2.55 (2H, t, J=7.2 Hz), 2.41-2.34 (2H, m), 2.24 (1H, dd, J=18.8, 10.0 Hz), 2.05-1.99 (1H, m), 1.81 (1H, brs), 1.73 (2H, qn, J=7.2 Hz), 1.58-1.25 (16H, m), 0.88 (3H, t, J=6.4 Hz).

### Example 14: 3,4,5-trimethoxyphenyl 7-((1R,2R,3R)-3-hydroxy-2-((S,E)-3-hydroxyoct-1-enyl)-5-oxocyclopentyl)heptanoate (Compound 14)

[chemical structure]

To a solution of 7-((1R,2R,3R)-3-hydroxy-2-((E)-3-hydroxyoct-1-enyl)-5-oxocyclopentyl)heptanoic acid (40.0 mg, 0.113 mmol) in DCM (2.0 mL) were successively added DMAP (13.8 mg, 0.113 mmol) and 3,4,5-trimethoxyphenol (29.1 mg, 0.158 mmol) at −10° C. under Ar atmosphere. The mixture was stirred at −10° C. for 3 minutes. After addition of EDC (30.3 mg, 0.158 mmol), the reaction mixture was stirred at room temperature for 14 hours. The mixture was purified by column chromatography on SiO₂ (EtOAc only) to afford 3,4,5-trimethoxyphenyl 7-((1R,2R,3R)-3-hydroxy-2-((S,E)-3-hydroxyoct-1-enyl)-5-oxocyclopentyl)heptanoate (48.6 mg, 83%) as a colorless oil.

¹H-NMR (400 MHz, CDCl₃): δ 6.32 (2H, s), 5.70 (1H, dd, J=14.7, 6.4 Hz), 5.57 (1H, dd, J=14.4, 8.4 Hz), 4.16-4.05 (2H, m), 3.84 (6H, s), 3.83 (3H, s), 2.76 (1H, dd, J=18.8, 7.2 Hz), 2.67 (1H, brs), 2.53 (2H, t, J=7.6 Hz), 2.41-2.34 (1H, m), 2.23 (1H, dd, J=18.8, 10.0 Hz), 2.05-1.99 (1H, m), 1.73 (2H, qn, J=7.2 Hz), 1.61-1.25 (17H, m), 0.89 (3H, t, J=6.8 Hz).

### Example 15: benzyl 7-((1R,2R,3R)-3-hydroxy-2-((S,E)-3-hydroxyoct-1-enyl)-5-oxocyclopentyl)heptanoate (Compound 15)

[chemical structure]

To a solution of 7-((1R,2R,3R)-3-hydroxy-2-((E)-3-hydroxyoct-1-enyl)-5-oxocyclopentyl)heptanoic acid (50.0 mg, 0.141 mmol) in DCM (2.0 mL) were successively added DMAP (17.2 mg, 0.141 mmol) and benzyl alcohol (20.5 μL, 0.197 mmol) at −10° C. under Ar atmosphere. The mixture was stirred at −10° C. for 3 minutes. After addition of EDC (37.9 mg, 0.197 mmol), the reaction mixture was stirred at room temperature for 14 hours. The mixture was purified by column chromatography on SiO₂ (EtOAc only) to afford benzyl 7-((1R,2R,3R)-3-hydroxy-2-((S,E)-3-hydroxyoct-1-enyl)-5-oxocyclopentyl)heptanoate (53.5 mg, 85%) as a colorless oil.

¹H-NMR (400 MHz, CDCl₃): δ 7.39-7.32 (5H, m), 5.71 (1H, dd, J=12.0, 6.8 Hz), 5.57 (1H, dd, J=14.4, 8.8 Hz), 5.11 (2H, s), 4.16-4.10 (2H, m), 2.75 (1H, dd, J=14.4, 8.0 Hz), 2.51 (1H, brs), 2.40-2.32 (3H, m), 2.23 (1H, dd, J=18.8, 9.6 Hz), 2.03-1.97 (1H, m), 1.62-1.27 (19H, m), 0.89 (3H, t, J=6.4 Hz).

### Examples 16 to 102: Compounds 16 to 102

Compounds 16 to 102 as shown in Tables 1 and 2 were synthesized referring to the methods disclosed in Examples 1 to 15.

### EXPERIMENTAL EXAMPLES

### Experimental Example 1: Structure-Based Design of PG Analogs

Cyclopentenone prostaglandin's A1/A2 are dehydrated metabolites of their precursors, the cyclopentanone prostaglandins E1/E2. In PGE1/E2, a hydroxyl group is attached to the C11 atom, which is responsible for the covalent attachment between PGA1 and Nurr1. This is evident from the electron density observed between the PGA1/A2's C11 atom (Rajan, S. et al. Nat Chem Biol 16, 876-886 (2020); U.S. patent application Ser. No. 16/334,550) and Nurr1's Cys566 sulphur, a characteristic feature of PGA1/A2 binding. Our NMR titration revealed that both PGA1 and PGE1 interact at same binding site on Nurr1-LBD corroborating with that in the crystal structure. Based on these one could conclude that both PGA1 and PGE1 can be used to design analogs, preferably using PGE1 which is better suited for chemic synthesis and modifications as it lacks the reactive C11 site, concealed by the hydroxyl group (Rajan, S. et al. Nat Chem Biol 16, 876-886 (2020)). In a similar way, addition of hydroxyl or methyl groups to PGE1 along the two hydrophobic tails has also been shown to prolong its metabolic degradation. One such is the misoprostol, which enhanced Nurr1's transcription function. Thus, we chose PGE1 and Misoprostol as the candidate molecules to make modification.

In this direction, the inventors of the present invention compared Nurr1-LBD crystal structures in the apo and PGA1-bound form. PGA1 interacts with Nurr1 residues Glu440, Phe443, Leu444, Arg515, His516, Arg563, Thr567, Cys566, Leu570, Ile573, Leu591, Phe592, Thr595 and Pro597 from helices 4, 11 and 12 (WO2018056905A1; U.S. patent application Ser. No. 16/334,550) constituting the primary binding site in Nurr1-LBD. A closer inspection revealed the presence of a ligand-induced surface pocket in Nurr1-LBD, lined by residues Glu445, Leu446, Leu509, Ala510, Thr513, Glu514, Arg515, Gln528, Val532, Leu556, Leu559, Pro560 and Arg563 from helices 9 and 11, forming the H9-H11 wedge. We have coined this pocket as the ‘secondary site’ and our hypothesis was to identify prostaglandin analogs, in which chemical fragments can be linked to the lead molecule, enabling it to occupy the secondary site and enhance the activity. The carboxyl group of PGA1 is oriented toward this secondary site and could serve as the point of modification for linking the fragment molecules. The secondary site could accommodate small fragments ranging between 100 and 120 Da. In this direction, a chemical synthesis protocol was adopted to link small fragments to the carboxyl (C1) end of PGE1 and misoprostol.

### Experimental Example 2: Activation of Nurr1-Mediated Transcription by the PG Analogs of the Present Invention

To investigate whether the PG analogs of the present invention activate Nurr1 function, the inventors of the present invention employed an reporter gene assay and examined the transcriptional activation as described in Kim, C.-H. et al., Proceedings of the National Academy of Sciences 112, 8756-8761, doi:10.1073/pnas.1509742112 (2015).

The gene assay was performed by Luciferase assay as described below:

[Protocol for Luciferase Assay to Determine Nurr1 Transcriptional Activity]
1. SK-N-BE2C cells were maintained in HyClone™ DMEM High Glucose (Cat. No.: SH30022.01) with 10% (v/v) fetal bovine serum (FBS), 100 units/ml penicillin and 100 μg/ml streptomycin.
2. Seed 1.5×10⁵ SK-N-BE2C cells/well into a 24-well plate and incubate at 37° C. with 5% CO₂ for 24 hours.
3. After 24 hours, transfect cells with pcDNA3.1 Myc/His mouse Nurr1 full-length construct, firefly luciferase reporter vector (pGL-3 basic from Promega, Cat. No.:E1751; with appropriate response element cloned in) and Renilla luciferase control vector (pRL-null from Promega, Cat. No.:E2271) in the ratio of 8:1:1 (Nurr1: firefly: renilla) or 400 ng, 50 ng and 50 ng respectively, amounting to a total of 500 ng in the 50 μl transfection mix for 1 well.
4. To make the transfection mix, first prepare the DNA with P3000 reagent by adding the appropriate amount of DNA with 1 μl P3000 reagent and top up with Opti-MEM® I Reduced-Serum Media (from ThermoFisher Scientific [Cat. No.: 31985070]) to 25 μl. Incubate for 5 minutes at room temperature.
5. Make another mix consisting of 1 μl Lipofectamine 3000 reagent with 24 μl Opti-MEM® I Reduced-Serum Media per well. Add this Lipofectamine mix to the previous DNA-P3000 mix (after the previous 5 minute incubation) and incubate them for 15 minutes at room temperature. [P3000:Lipofectamine=1:1].
6. In the meantime, change the media in the sample wells with fresh DMEM media without antibiotics.
7. After 15 minutes incubation, add the 50 μl transfection mix into each well and incubate the plate for 24 hours at 37° C. with 5% CO₂.
8. After 24 hours, change the media to normal DMEM media with antibiotics and add various doses of compounds into designated wells and incubate the plate for another 24 hours at 37° C. with 5% CO₂.
9. Following the 24-hour incubation period, luciferase assay can be conducted using Dual-Glo Luciferase® kit from Promega (Cat. No.: E2920).
10. Lyse each tested wells for 15 minutes with 75 μl of passive lysis buffer provided in the Dual-Luciferase® kit from Promega (Cat. No.: E1910). [Alternatively, add 75 μl of PBS and 75 μl of Dual-Glo® Luciferase Reagent and incubate for 10 minutes at room temperature (cover the plate with foil to protect from light). Transfer the entire sample in each well to a Greiner CELLSTAR® 96-well white polystyrene plate then proceed to step 13].
11. After 15 minutes, transfer each lysed sample to a Greiner CELLSTAR® 96-well white polystyrene plate (Cat. No.:655083).
12. To each well of lysed cells, add 75 μl of Dual-Glo® Luciferase Reagent and incubate for 10 minutes at room temperature (cover the plate with foil to protect from light).
13. Set the measurement with a 10-second integrated measurement period.
14. Add 75 μl of Dual-Glo® Stop & Glo® reagent into each well and incubate for 10 minutes before commencing with the same parameters as before to determine the RLU for the Renilla luciferase.
15. The averaged reading for wells corresponding to non-transfected cells can be subtracted from each RLU reading and duplicate/triplicate readings can then be averaged. Normalization of the luminescence reading for each well can be carried out by dividing the firefly luciferase RLU with the corresponding Renilla luciferase RLU. Each drug-treated well can then be normalized against the averaged DMSO-treated well reading.

The results are shown in below Table 3. Notably, tested PG analogs of the present invention stimulated Nurr1-dependent transcriptional activity through its LBD in a dose-dependent manner as they induced Nurr1 LBD-based reporter activity up to 0.5˜2.9-fold:

TABLE 3
Transcription Assays Data
Fold activity: Relative activity over control (luminescence
value; 1.0) A: above 2.0 (high activity), B: 1.1~2.0
(moderate), C: below 1.0 (low activity)

| Comp. | Fold activity | |
|---|---|---|
| # | (1 uM) | |
| 1 | A | Example 1 |
| 2 | A | Example 2 |
| 3 | B | Example 3 |
| 4 | A | Example 4 |
| 5 | A | Example 5 |
| 6 | B | Example 6 |
| 7 | A | Example 7 |
| 8 | A | Example 8 |
| 9 | A | Example 9 |
| 10 | A | Example 10 |
| 11 | A | Example 11 |
| 12 | A | Example 12 |
| 13 | A | Example 13 |
| 14 | A | Example 14 |
| 15 | A | Example 15 |
| 16 | A | 15(R)-Prostaglandin D2 |
| 17 | B | Prostaglandin D2-1-glyceryl ester |
| 18 | A | Prostaglandin E1 Ethanolamide |
| 19 | B | 15-keto Prostaglandin E1 |
| 20 | A | Prostaglandin E2 |
| 21 | B | 11b-Prostaglandin E2 |
| 22 | B | Sulprostone |
| 23 | B | 11-keto Fluprostenol |
| 24 | A | Prostaglandin E2-1-glyceryl ester |
| 25 | B | 11-deoxy-11-methylene Prostaglandin |
| | | D2 |
| 26 | A | Prostaglandin E1 Alcohol |
| 27 | A | 15(S)-15-methyl Prostaglandin E1 |
| 28 | A | Prostaglandin E2 methyl ester |
| 29 | B | 13,14-dihydro-15-keto Prostaglandin |
| | | E2 |
| 30 | A | 16-phenyl tetranor Prostaglandin E2 |
| 31 | B | (R)-Butaprost (free acid) |
| 32 | C | Prostaglandin D2 serinol amide |
| 33 | B | 13,14-dihydro-15-keto Prostaglandin |
| | | D2 |
| 34 | B | 1a,1b-dihomo Prostaglandin E1 |
| 35 | B | (R)-Butaprost |
| 36 | B | Prostaglandin E2 Ethanolamide |
| 37 | B | 15(R)-Prostaglandin E2 |
| 38 | B | 17-phenyl trinor Prostaglandin E2 |
| 39 | B | Prostaglandin E2-biotin |
| 40 | C | Prostaglandin E2 serinol amide |
| 41 | C | D12-Prostaglandin D2 |
| 42 | A | 6-keto Prostaglandin E1 |
| 43 | B | CAY10408 |
| 44 | A | Prostaglandin E2 p-acetamidophenyl |
| | | ester |
| 45 | B | 15-keto Prostaglandin E2 |
| 46 | C | tetranor-PGEM |
| 47 | B | ent-Prostaglandin E2 |
| 48 | C | Prostaglandin D1 |
| 49 | C | 15(R)-15-methyl Prostaglandin D2 |
| 50 | A | 8-iso Prostaglandin E1 |
| 51 | A | 16,16-dimethyl Prostaglandin E1 |
| 52 | A | Prostaglandin E2 p-benzamidophenyl |
| | | ester |
| 53 | B | 15(R)-15-methyl Prostaglandin E2 |
| 54 | A | 19(R)-hydroxy Prostaglandin E2 |
| 55 | C | Prostaglandin D2 methyl ester |
| 56 | C | Prostaglandin D1 Alcohol |
| 57 | B | 15(S)-15-methyl Prostaglandin D2 |
| 58 | A | 13,14-dihydro Prostaglandin E1 |
| 59 | B | 16-phenyl tetranor Prostaglandin E1 |
| 60 | B | 5-trans Prostaglandin E2 |
| 61 | A | 15(S)-15-methyl Prostaglandin E2 |
| 62 | A | 20-ethyl Prostaglandin E2 |
| 63 | B | 3-methoxy Limaprost |
| 64 | B | Prostaglandin D2 |
| 65 | C | 17-phenyl trinor Prostaglandin D2 |
| 66 | B | 13,14-dihydro-15(R)-Prostaglandin E1 |
| 67 | A | Limaprost |
| 68 | A | 8-iso Prostaglandin E2 |
| 69 | A | 16,16-dimethyl Prostaglandin E2 |
| 70 | A | Prostaglandin E3 |
| 71 | B | 8-iso-16-cyclohexyl-tetranor |
| | | Prostaglandin E2 |
| 72 | C | Prostaglandin D2 Ethanolamide |
| 73 | A | Prostaglandin E1 |
| 74 | B | 13,14-dihydro-15-keto Prostaglandin |
| | | E1 |
| 75 | A | Misoprostol |
| 76 | A | 8-iso Prostaglandin E2 isopropyl ester |
| 77 | A | 16,16-dimethyl Prostaglandin E2 p-(p- |
| | | acetamidobenzamido) phenyl ester |
| 78 | A | 17-trans Prostaglandin E3 |
| 79 | C | 13,14-dihydro-15-keto Prostaglandin |
| | | D1 |
| 80 | C | 17-phenyl trinor PGF2a methyl amide |
| 81 | C | 11β-Misoprostol |
| 82 | B | 8-iso-15-keto Prostaglandin E2 |
| 83 | B | 19(R)-hydroxy Prostaglandin E1 |
| 84 | B | 16,16-dimethyl-6-keto Prostaglandin |
| | | E1 |
| 85 | C | 11b-Prostaglandin E1 |
| 86 | C | Carboprost |
| 87 | C | 15(R)-PGE1 |
| 88 | B | Alprostadil Ethanolamide |
| 89 | A | 1,11a,15S-Trihydroxy-prost-13E-EN- |
| | | 9-ONE |
| 90 | A | BW245C |
| 91 | B | Latanoprost |
| 92 | B | DinoprostTromethanol |
| 93 | C | 2,3-DINOR PGE1 |
| 94 | B | Misoprostol acid |
| 95 | C | Latanoprostene bunod |
| 96 | C | — |
| 97 | B | — |
| 98 | — | — |
| 99 | — | Enprostil |
| 100 | — | Rioprostol |
| 101 | — | Ornoprostol |
| 102 | — | Gemeprost |

### Experimental Example 3: Structural Models of PG Analogs Bound to Nurr1-LBD

The inventors of the present invention then modelled the two PG analogs, Compound 1 and Compound 2 (FIGS. 1A and 1B), and docked them with Nurr1-LBD, using our earlier PGA1-bound Nurr1-LBD (PDB ID 5Y41) as the starting model. The fragment (benzylamino phenyl ester) was built and linked with PGA1 using the PyMOL software, to generate the Table Compound #1 PG analog. While PGA1 was modified to misoprostol and the same fragment was attached, to build the Table compound #2 molecule (FIGS. 1A and B). Manual docking of these PG analogs into Nurr1-LBD, guided by the Nurr1-LBD-PGA1 crystal structure, was followed by 1000 cycles of energy minimization using the ‘Minimize Structure’ module using the software Chimera, to remove any short-contacts between the protein and ligand atoms, if any. The resulting models revealed that the linked fragments fit well into the secondary site making key hydrogen bonded interactions with backbone atoms of Pro560 and Arg563, and the side chain of Thr513, as well as non-polar contacts with other neighbouring residues (FIG. 1C). In addition, the modifications at the C16 position in Compound 2 also accommodates itself within the groove in Nurr1-LBD, stabilized by key hydrogen bonded interactions with Glu440, Leu591 and Thr595 (FIG. 1D). The interactions reveal that the additional modifications enhance the interactions with the nearby protein atoms (FIGS. 1C and 1D).

### Experimental Example 4: Intranasal Administration of the Present Invention for Treating 6-OHDA Parkinson's Disease Model

Mice were randomly divided into four groups: Control (n=4); 6-OHDA (6-hydroxydopamine)+(n=7), 6-OHDA+PGE1 (n=8); 6-OHDA+BSC15 (the compound of the present invention) (n=8); 6-OHDA+BSC19 (the compound of the present invention) (n=7).

Week 9-10 C57BL/6 mice were intranasally administered daily with 0.2 mg/ml of compounds to be tested for 3 days before 6-OHDA injection. Stereotaxic surgery was performed by injecting 7.5 μg of 6-OHDA into the left striatum (AP: +0.5 mm, ML: −2.0 mm, VD: −3.5 mm) of the mouse. Compounds to be tested, i.e., PGE1, BSC15 and BSC19 were prepared by double emulsion solvent evaporation of 1:4 molar ratio with 2-hydroxypropyl-β-cyclodextrin. After surgical recovery, the daily intranasal administration continued with a weekly assessment of apomorphine (0.5 mg/kg) induced rotational behavior. Animal care and handling were performed according to the protocol approved by Nanyang Technological University's Institutional Animal Care and Use Committee.

Mice treated with PGE1, BSC15 and BSC19 showed a significant reduction in rotation number, suggesting a substantial recovery from the 6-OHDA-induced behavioral deficit. (FIG. 2A) Among the three compounds, the BSC15-treated group displayed remarkable recovery, where the group average rotation number decreased by 96% in 3 weeks of administration. To validate that the compounds did not have any adverse effects, we monitored the compound treated mice's body weight and observed that they were similar to the control groups (FIG. 2B). These findings suggest that the PGE1 derivatives BSC15 and BSC19 could be considered to be developed to treat Parkinson's disease as a neuroprotective agent.

### CONCLUSION

As shown above, the PG analogs of the present invention could enhance the activity of Nurr1, and show remarkable effect in treating Parkinson's disease. The invention can be expanded to cancer, rheumatoid arthritis, Alzheimers disease, schizophrenia, manic depression or any disease, disorder, or condition associated with Nurr1. Systematic approach from structure to synthesis and characterization has been provided here to claim that the PG analogs of the present invention could serve as potent therapeutic agents toward treatment of a disease, disorder, or condition associated with Nurr1.

1. A prostaglandin analog represented by the following Chemical Formula I, or a pharmaceutically acceptable salt, stereoisomer, solvate, polymorph, ester, tautomer, or prodrug thereof. [chemical structure]
wherein,
X is non-substituted or substituted —(C1-C8)alkyl, —[(C1-C8)alkoxy](C1-C8)alkyl-, —(C1-C8)alkylcarboxylic acid —(C1-C8)alkylcarboxylester, —(C1-C8)akenyl, [(C1-C8)alkoxy](C1-C8)alkenyl, —(C1-C8)alkenyl acid, —(C1-C8)alkenyl ester, —(C1-C8)alkylamide, or —(C1-C8)alkenylamide;
Y is (C1-C8) alkyl or (C1-C8) alkenyl, which is optionally substituted with one or more substituent(s) selected from the group consisting of hydroxy, oxo, halo, (C1-C6)alkyl, (C1-C6)alkoxy, (C6-C10) aryl, (C6-C10) aryloxy being optionally substituted with (C1-C3) alkyl or halo(C1-C3) alkyl, (C3-C10) cycloalkyl;
A₁ and A₂ are each independently, CH, CH₂, NH or N;
Z is ═O, ═CH₂, [symbol] , or [symbol] ;
Z″ is ═O, ═CH₂, [symbol] , or [symbol] , R_{d} is H, (C1-C3) alkyl, (C1-C6)acylcarbonyl or tetrahydropyranyl,
in the Chemical Formula, the notation [symbol] is a single bond or a double bond.

2. The prostaglandin analog, or a pharmaceutically acceptable salt, stereoisomer, solvate, polymorph, ester, tautomer, or prodrug thereof according to claim 1,
wherein X is —(C1-C8) alkyl or —(C1-C8) alkenyl which is optionally substituted with one or more substituents selected from the group consisting of hydroxyl, —(C1-C6) alkoxy, —C(═O)ORₐ, —C(═O)NRₐR_{b},
where Rₐ is H, (C1-C8) alkyl, (C6-C9) aryl, (C6-C9) aryloxy, —NH(C6-C9)aryl, 5- to 12-membered heteroaryl having one or more heteroatom selected from the group consisting of N, O and S, said (C1-C8) alkyl, (C6-C9) aryl, 5- to 12-membered heteroaryl may be optionally substituted with halo, hydroxyl, cyano, nitro, amino, substituted amino, (C1-C6)acyl, —ONO₂, (C1-C8) alkoxy, (C1-C8)alkyl, substituted (C1-C8)alkyl, (C1-C8)haloalkyl, (C3-C7)cycloalkyl, (C1-C8)alkylcarboxy, —NHC(═O)R_{c}, or —C(═O)R_{c}, where R_{c} is (C1-C8) alkyl or (C6-C9) aryl which may be optionally substituted with one or more substituents of halo, CF₃, (C1-C6)acyl, amino, substituted amino, cyano, nitro, (C1-C8)alkyl, substituted (C1-C8)alkyl, (C1-C8)haloalkyl, (C1-C8)alkoxy, (C1-C3)acyloxy, and (C6-C9)aryloxy 5- to 12-membered heterocycloalkyl having one or more heteroatoms selected from the group consisting of N, O and S;
and
R_{b} is H or —(C1-C6)alkyl.

3. The prostaglandin analog, or a pharmaceutically acceptable salt, stereoisomer, solvate, polymorph, ester, tautomer, or prodrug thereof according to claim 2,
wherein Rₐ is one selected from the group consisting of below substituents:
H, —CH₃, —CH₂CH₃, —CH(CH₃)₂, —CH₂CH₂OH, —CH₂CH(OH)CH₂OH, —CH(CH₂OH)₂, —SO₂CH₃, [chemical structure] [chemical structure]
and
R_{b} is H or —(C1-C3)alkyl.

4. The prostaglandin analog, or a pharmaceutically acceptable salt, stereoisomer, solvate, polymorph, ester, tautomer, or prodrug thereof according to claim 1,
wherein, X is —(C1-C8)alkyl-OH, —(C1-C8)alkyl-O—(C1-C6)alkyl, —(C1-C8)alkyl-CO₂H, —(C1-C8)alkenyl-CO₂H, —(C1-C8)alkyl-CO₂—(C1-C6)alkyl, —(C1-C8)alkyl-CO₂R², —(C1-C8)alkenyl-CO₂R², —(C1-C8)alkyl-CONR³R⁴, —(C1-C8)alkyl-CONHOR⁴, —(C1-C8)alkenyl-CONR³R⁴, or —(C1-C8)alkyl-CONHOR⁴),
where R² is —(C1-C6)alkyl, Ar, CH₂Ar, —Ar—NHCO—Ar, or —Ar—CONH—Ar, said —(C1-C6)alkyl is optionally with one to three substituents selected from the group consisting of (C1-C6) alkyl, hydroxyl, halogen (C1-C6)alkoxy or CF3;
R³ is H or —(C1-C6)alkyl;
R⁴ is H, —(C1-C6)alkyl, Ar, Ar—NHCO—Ar, or Ar—CONH—Ar; and
Ar is a (C6-C10) aryl, 5- to 12 membered heteroaryl, or hetero-biaryl, which is optionally substituted with one or more substituents independently selected from the group consisting of (C1-C6)alkoxy, halogen, (C1-C6)alkyl, and CF₃.

5. The prostaglandin analog, or a pharmaceutically acceptable salt, stereoisomer, solvate, polymorph, ester, tautomer, or prodrug thereof according to claim 1,
wherein X is —CH₂CH₂CH₂CH₂CH₂CH₂OH, —CH₂CH₂CH₂CH₂CH₂CH₂OCH₃, —CH₂CH₂CH₂CH₂CH₂CH₂CO₂H, —CH═CHCH₂CH₂CH₂CO₂H, —CH₂CH₂CH₂CH₂CH₂CO₂CH₃, —CH₂CH₂CH₂CH₂CH₂CO₂CH₂CH₃, —CH₂CH₂CH₂CH₂CH₂CO₂R², —CH═CHCH₂CH₂CH₂CO₂R², —CH₂CH₂CH₂CH₂CH₂CONR³R⁴, —CH₂CH₂CH₂CH₂CH₂CONHOR⁴, —CH═CHCH2CH2CH2CONR³R⁴, or —CH═CHCH₂CH₂CH₂CONHOR⁴.

6. The prostaglandin analog, or a pharmaceutically acceptable salt, stereoisomer, solvate, polymorph, ester, tautomer, or prodrug thereof according to claim 1,
wherein Y is (C1-C6) alkyl or (C1-C6) alkenyl, which is optionally substituted with one to three substituent(s) selected from the group consisting of hydroxy, oxo, halo, methyl, methoxy, phenyl, phenoxy being optionally substituted with CF₃, cylobutyl and cyclohexyl.

7. The prostaglandin analog, or a pharmaceutically acceptable salt, stereoisomer, solvate, polymorph, ester, tautomer, or prodrug thereof according to claim 1,
wherein, the prostaglandin analog of Chemical Formula I is represented by Formula II: [chemical structure]
wherein X, Y and R_{d} are the same as defined in Chemical formula I of claim 1.

8. The prostaglandin analog, or a pharmaceutically acceptable salt, stereoisomer, solvate, polymorph, ester, tautomer, or prodrug thereof according to claim 1,
wherein, the prostaglandin analog of Chemical Formula I is represented by one of Chemical Formula IIIa, IIIb, IIIc, IIId, IIIe and IIIf: [chemical structure]
wherein;
R¹ is CH₂OH, CH₂OCH₃, CO₂H, CO₂CH₃, CO₂CH₂CH₃, CO₂R², CONR³R⁴, or CONHOR⁴;
R² is —(C1-C6)alkyl, Ar, CH₂Ar, —Ar—NHCO—Ar, or —Ar—CONH—Ar, said —(C1-C6)alkyl is optionally with one to three substituents selected from the group consisting of (C1-C6) alkyl, hydroxyl, halogen (C1-C6)alkoxy or CF3;
R³ is H, —(C1-C6)alkyl;
R⁴ is H, —(C1-C6)alkyl, Ar, Ar—NHCO—Ar, or Ar—CONH—Ar;
Ar is a (C6-C10) aryl, 5- to 12 membered heteroaryl, or hetero-biaryl, which is optionally substituted with one or more substituents independently selected from the group consisting of (C1-C6)alkoxy, halogen, (C1-C6)alkyl, and CF₃.

9. The prostaglandin analog, or a pharmaceutically acceptable salt, stereoisomer, solvate, polymorph, ester, tautomer, or prodrug thereof according to claim 1,
wherein, the prostaglandin analog of Chemical Formula I is represented by one of Chemical Formula IVa, IVb, IVc, and IVd, as below: [chemical structure]
wherein,
Y is —(C1-C6)alkyl, —(C1-C6)fluoroalkyl, —(C1-C6)difluoroalkyl, —(C1-C6)trifluoroalkyl, —(C1-C6)hydroxyalkyl, —(C2-C6)dihydroxyalkyl, or [(C1-C6)alkoxy](C1-C6)alkyl, which is optionally substituted with one to three substituents selected from the group consisting of (C1-C6) alkyl, halogen, hydroxyl, (C1-C6)alkoxy, or CF3.
each R⁵ is independently selected from the group consisting of hydrogen, halogen, CF3, (C1-C6)acyl, amino, substituted amino, (C1-C6)alkyl, substituted (C1-C6)alkyl, (C1-C6)haloalkyl, (C3-C7)cycloalkyl, (C1-C6)alkylcarboxy, cyano, nitro, and (C1-C6)alkoxy,
each R⁶ is independently selected from the group consisting of hydrogen, halogen, CF3, (C1-C10)acyl, amino, substituted amino, (C1-C6)alkyl, substituted (C1-C6)alkyl, (C1-C6)haloalkyl, cyano, nitro, (C1-C6)alkoxy, (C1-C3)acyloxy, and (C6-C10)aryloxy; and
n is 0, 1, 2, 3, 4 or 5.

10. The prostaglandin analog, or a pharmaceutically acceptable salt, stereoisomer, hydrate, solvate or prodrug thereof according to claim 1, wherein the prostaglandin analog is selected from the group consisting of the following compounds 2 to 15, 96 and 97:

| Compound # | Structure |
|---|---|
| 2 | [chemical structure] |
| 3 | [chemical structure] |
| 4 | [chemical structure] |
| 5 | [chemical structure] |
| 6 | [chemical structure] |
| 7 | [chemical structure] |
| 8 | [chemical structure] |
| 9 | [chemical structure] |
| 10 | [chemical structure] |
| 11 | [chemical structure] |
| 12 | [chemical structure] |
| 13 | [chemical structure] |
| 14 | [chemical structure] |
| 15 | [chemical structure] |
| 96 | [chemical structure] |
| 97 | [chemical structure] |

11. The prostaglandin analog, or a pharmaceutically acceptable salt, stereoisomer, hydrate, solvate or prodrug thereof according to claim 1, wherein the prostaglandin analog is not the compound selected from the group consisting of the following compounds 1, 16 to 95, and 98 to 102:

| Compound # | Structure |
|---|---|
| 1 | [chemical structure] |
| 16 | [chemical structure] |
| 17 | [chemical structure] |
| 18 | [chemical structure] |
| 19 | [chemical structure] |
| 20 | [chemical structure] |
| 21 | [chemical structure] |
| 22 | [chemical structure] |
| 23 | [chemical structure] |
| 24 | [chemical structure] |
| 25 | [chemical structure] |
| 26 | [chemical structure] |
| 27 | [chemical structure] |
| 28 | [chemical structure] |
| 29 | [chemical structure] |
| 30 | [chemical structure] |
| 31 | [chemical structure] |
| 32 | [chemical structure] |
| 33 | [chemical structure] |
| 34 | [chemical structure] |
| 35 | [chemical structure] |
| 36 | [chemical structure] |
| 37 | [chemical structure] |
| 38 | [chemical structure] |
| 39 | [chemical structure] |
| 40 | [chemical structure] |
| 41 | [chemical structure] |
| 42 | [chemical structure] |
| 43 | [chemical structure] |
| 44 | [chemical structure] |
| 45 | [chemical structure] |
| 46 | [chemical structure] |
| 47 | [chemical structure] |
| 48 | [chemical structure] |
| 49 | [chemical structure] |
| 50 | [chemical structure] |
| 51 | [chemical structure] |
| 52 | [chemical structure] |
| 53 | [chemical structure] |
| 54 | [chemical structure] |
| 55 | [chemical structure] |
| 56 | [chemical structure] |
| 57 | [chemical structure] |
| 58 | [chemical structure] |
| 59 | [chemical structure] |
| 60 | [chemical structure] |
| 61 | [chemical structure] |
| 62 | [chemical structure] |
| 63 | [chemical structure] |
| 64 | [chemical structure] |
| 65 | [chemical structure] |
| 66 | [chemical structure] |
| 67 | [chemical structure] |
| 68 | [chemical structure] |
| 69 | [chemical structure] |
| 70 | [chemical structure] |
| 71 | [chemical structure] |
| 72 | [chemical structure] |
| 73 | [chemical structure] |
| 74 | [chemical structure] |
| 75 | [chemical structure] |
| 76 | [chemical structure] |
| 77 | [chemical structure] |
| 78 | [chemical structure] |
| 79 | [chemical structure] |
| 80 | [chemical structure] |
| 81 | [chemical structure] |
| 82 | [chemical structure] |
| 83 | [chemical structure] |
| 84 | [chemical structure] |
| 85 | [chemical structure] |
| 86 | [chemical structure] |
| 87 | [chemical structure] |
| 88 | [chemical structure] |
| 89 | [chemical structure] |
| 90 | [chemical structure] |
| 91 | [chemical structure] |
| 92 | [chemical structure] |
| 93 | [chemical structure] |
| 94 | [chemical structure] |
| 95 | [chemical structure] |
| 98 | [chemical structure] |
| 99 | [chemical structure] |
| 100 | [chemical structure] |
| 101 | [chemical structure] |
| 102 | [chemical structure] |

12. A pharmaceutical composition for modulating Nurr1, comprising a prostaglandin analog of Chemical Formula I according to claim 1, or a pharmaceutically acceptable salt, stereoisomer, solvate, polymorph, ester, tautomer, or prodrug thereof, as an active ingredient, and a pharmaceutically acceptable carrier.

13. The pharmaceutical composition of claim 12, wherein the modulation of Nurr1 is activation of Nurr1.

14. A pharmaceutical composition for preventing or treating a disease, disorder, or condition associated with Nurr1, comprising a prostaglandin analog of Chemical Formula I according to claim 1, or a pharmaceutically acceptable salt, stereoisomer, solvate, polymorph, ester, tautomer, or prodrug thereof, as an active ingredient, and a pharmaceutically acceptable carrier:

15. The pharmaceutical composition of anyone of claims 12 to 14, wherein the prostaglandin analog is selected from the group consisting of the following compounds 1 to 102:

| Compound # | Structure |
|---|---|
| 1 | [chemical structure] |
| 2 | [chemical structure] |
| 3 | [chemical structure] |
| 4 | [chemical structure] |
| 5 | [chemical structure] |
| 6 | [chemical structure] |
| 7 | [chemical structure] |
| 8 | [chemical structure] |
| 9 | [chemical structure] |
| 10 | [chemical structure] |
| 11 | [chemical structure] |
| 12 | [chemical structure] |
| 13 | [chemical structure] |
| 14 | [chemical structure] |
| 15 | [chemical structure] |
| 16 | [chemical structure] |
| 17 | [chemical structure] |
| 18 | [chemical structure] |
| 19 | [chemical structure] |
| 20 | [chemical structure] |
| 21 | [chemical structure] |
| 22 | [chemical structure] |
| 23 | [chemical structure] |
| 24 | [chemical structure] |
| 25 | [chemical structure] |
| 26 | [chemical structure] |
| 27 | [chemical structure] |
| 28 | [chemical structure] |
| 29 | [chemical structure] |
| 30 | [chemical structure] |
| 31 | [chemical structure] |
| 32 | [chemical structure] |
| 33 | [chemical structure] |
| 34 | [chemical structure] |
| 35 | [chemical structure] |
| 36 | [chemical structure] |
| 37 | [chemical structure] |
| 38 | [chemical structure] |
| 39 | [chemical structure] |
| 40 | [chemical structure] |
| 41 | [chemical structure] |
| 42 | [chemical structure] |
| 43 | [chemical structure] |
| 44 | [chemical structure] |
| 45 | [chemical structure] |
| 46 | [chemical structure] |
| 47 | [chemical structure] |
| 48 | [chemical structure] |
| 49 | [chemical structure] |
| 50 | [chemical structure] |
| 51 | [chemical structure] |
| 52 | [chemical structure] |
| 53 | [chemical structure] |
| 54 | [chemical structure] |
| 55 | [chemical structure] |
| 56 | [chemical structure] |
| 57 | [chemical structure] |
| 58 | [chemical structure] |
| 59 | [chemical structure] |
| 60 | [chemical structure] |
| 61 | [chemical structure] |
| 62 | [chemical structure] |
| 63 | [chemical structure] |
| 64 | [chemical structure] |
| 65 | [chemical structure] |
| 66 | [chemical structure] |
| 67 | [chemical structure] |
| 68 | [chemical structure] |
| 69 | [chemical structure] |
| 70 | [chemical structure] |
| 71 | [chemical structure] |
| 72 | [chemical structure] |
| 73 | [chemical structure] |
| 74 | [chemical structure] |
| 75 | [chemical structure] |
| 76 | [chemical structure] |
| 77 | [chemical structure] |
| 78 | [chemical structure] |
| 79 | [chemical structure] |
| 80 | [chemical structure] |
| 81 | [chemical structure] |
| 82 | [chemical structure] |
| 83 | [chemical structure] |
| 84 | [chemical structure] |
| 85 | [chemical structure] |
| 86 | [chemical structure] |
| 87 | [chemical structure] |
| 88 | [chemical structure] |
| 89 | [chemical structure] |
| 90 | [chemical structure] |
| 91 | [chemical structure] |
| 92 | [chemical structure] |
| 93 | [chemical structure] |
| 94 | [chemical structure] |
| 95 | [chemical structure] |
| 96 | [chemical structure] |
| 97 | [chemical structure] |
| 98 | [chemical structure] |
| 99 | [chemical structure] |
| 100 | [chemical structure] |
| 101 | [chemical structure] |
| 102 | [chemical structure] |

16. The pharmaceutical composition of claim 14, wherein the a disease, disorder, or condition associated with Nurr1 is selected from the group consisting of cancer, autoimmune disease, schizophrenia, manic depression and neurodegenerative disease.

17. The pharmaceutical composition of claim 16, wherein the autoimmune disease is rheumatoid arthritis, and the neurodegenerative disease is Alzheimers disease or Parkinson's disease.

18. A method of modulating Nurr1, comprising administering an effective amount of the prostaglandin analog of Chemical Formula I according to claim 1, or a pharmaceutically acceptable salt, stereoisomer, solvate, polymorph, ester, tautomer, or prodrug thereof, to a subject in need of modulating Nurr1.

19. A method of for preventing or treating a disease, disorder, or condition associated with Nurr1, comprising administering an effective amount of the prostaglandin analog of Chemical Formula I according to claim 1, or a pharmaceutically acceptable salt, stereoisomer, solvate, polymorph, ester, tautomer, or prodrug thereof, to a subject in need of preventing or treating a disease, disorder, or condition associated with Nurr1.

20. The method of claim 19, wherein the a disease, disorder, or condition associated with Nurr1 is selected from the group consisting of cancer, rheumatoid arthritis, Alzheimers disease, schizophrenia, manic depression and Parkinson's disease.

21. The method of claim 20, wherein the autoimmune disease is rheumatoid arthritis, and the neurodegenerative disease is Alzheimers disease or Parkinson's disease.

21. The method of anyone of claims 18 to 21, wherein the prostaglandin analog is selected from the group consisting of the following compounds 1 to 102:

| Compound # | Structure |
|---|---|
| 1 | [chemical structure] |
| 2 | [chemical structure] |
| 3 | [chemical structure] |
| 4 | [chemical structure] |
| 5 | [chemical structure] |
| 6 | [chemical structure] |
| 7 | [chemical structure] |
| 8 | [chemical structure] |
| 9 | [chemical structure] |
| 10 | [chemical structure] |
| 11 | [chemical structure] |
| 12 | [chemical structure] |
| 13 | [chemical structure] |
| 14 | [chemical structure] |
| 15 | [chemical structure] |
| 16 | [chemical structure] |
| 17 | [chemical structure] |
| 18 | [chemical structure] |
| 19 | [chemical structure] |
| 20 | [chemical structure] |
| 21 | [chemical structure] |
| 22 | [chemical structure] |
| 23 | [chemical structure] |
| 24 | [chemical structure] |
| 25 | [chemical structure] |
| 26 | [chemical structure] |
| 27 | [chemical structure] |
| 28 | [chemical structure] |
| 29 | [chemical structure] |
| 30 | [chemical structure] |
| 31 | [chemical structure] |
| 32 | [chemical structure] |
| 33 | [chemical structure] |
| 34 | [chemical structure] |
| 35 | [chemical structure] |
| 36 | [chemical structure] |
| 37 | [chemical structure] |
| 38 | [chemical structure] |
| 39 | [chemical structure] |
| 40 | [chemical structure] |
| 41 | [chemical structure] |
| 42 | [chemical structure] |
| 43 | [chemical structure] |
| 44 | [chemical structure] |
| 45 | [chemical structure] |
| 46 | [chemical structure] |
| 47 | [chemical structure] |
| 48 | [chemical structure] |
| 49 | [chemical structure] |
| 50 | [chemical structure] |
| 51 | [chemical structure] |
| 52 | [chemical structure] |
| 53 | [chemical structure] |
| 54 | [chemical structure] |
| 55 | [chemical structure] |
| 56 | [chemical structure] |
| 57 | [chemical structure] |
| 58 | [chemical structure] |
| 59 | [chemical structure] |
| 60 | [chemical structure] |
| 61 | [chemical structure] |
| 62 | [chemical structure] |
| 63 | [chemical structure] |
| 64 | [chemical structure] |
| 65 | [chemical structure] |
| 66 | [chemical structure] |
| 67 | [chemical structure] |
| 68 | [chemical structure] |
| 69 | [chemical structure] |
| 70 | [chemical structure] |
| 71 | [chemical structure] |
| 72 | [chemical structure] |
| 73 | [chemical structure] |
| 74 | [chemical structure] |
| 75 | [chemical structure] |
| 76 | [chemical structure] |
| 77 | [chemical structure] |
| 78 | [chemical structure] |
| 79 | [chemical structure] |
| 80 | [chemical structure] |
| 81 | [chemical structure] |
| 82 | [chemical structure] |
| 83 | [chemical structure] |
| 84 | [chemical structure] |
| 85 | [chemical structure] |
| 86 | [chemical structure] |
| 87 | [chemical structure] |
| 88 | [chemical structure] |
| 89 | [chemical structure] |
| 90 | [chemical structure] |
| 91 | [chemical structure] |
| 92 | [chemical structure] |
| 93 | [chemical structure] |
| 94 | [chemical structure] |
| 95 | [chemical structure] |
| 96 | [chemical structure] |
| 97 | [chemical structure] |
| 98 | [chemical structure] |
| 99 | [chemical structure] |
| 100 | [chemical structure] |
| 101 | [chemical structure] |
| 102 | [chemical structure] |
